(19) United States
(12) Patent Application Publication
(10) Pub. No.: US 2018/0000362 A1
(43) Pub. Date: Jan. 4, 2018

(54) BIOLOGICAL INFORMATION MEASURING MODULE AND BIOLOGICAL INFORMATION MEASURING APPARATUS

(71) Applicant: SEIKO EPSON CORPORATION, Tokyo (JP)

(72) Inventors: Atsushi MATSUO, Azumino-shi (JP); Akira INAGAKI, Matsumoto-shi (JP)

(73) Assignee: SEIKO EPSON CORPORATION, Tokyo (JP)

(21) Appl. No.: 15/617,651

(22) Filed: Jun. 8, 2017

(30) Foreign Application Priority Data
Jul. 1, 2016 (JP) 2016-131382

(51) Int. Cl. A61B 5/024; A61B 5/00

(52) U.S. Cl. CPC A61B 5/02427; A61B 5/7455; A61B 5/681; A61B 2562/0238; A61B 2562/043; A61B 2562/185; A61B 2562/028

(57) ABSTRACT

A biological information measuring module includes light emitters (first light emitter and second light emitter), a reflector having a tapered section that inclines with respect to the direction in which light emitted from the light emitters travels toward a target, and a light receiver that receives reflected light that is light exits from the light emitters and via the reflector and is reflected off the target.

### CROSS-REFERENCES TO RELATED APPLICATIONS

This application claims priority to Japanese Patent Application No. 2016-131382, filed Jul. 1, 2016, the entirety of which is herein incorporated by reference.

### BACKGROUND

### 1. Technical Field

The present invention relates to a biological information measuring module and a biological information measuring apparatus.

### 2. Related Art

There is a known biological information measuring module that is worn around a wrist or any other body site of a wearer with the aid of a band or any other component and measures the wearer's biological information, such as pulse waves, and there is a known wristwatch-shaped wrist apparatus having the function of measuring the biological information (biological information measuring apparatus). For example, WO 2014/091424 discloses a biological information measuring apparatus that is worn around a wearer's (subject's) arm (wrist) as a target of the measurement and incorporates a biological information measuring module that measures biological information, such as pulse waves, with an optical sensor.

The biological information measuring module using an optical sensor and the biological information measuring apparatus using the biological information measuring module optically measure blood flow under a skin surface that is a target of the measurement and convert a result of the measurement into a signal to produce biological information, such as pulse waves. it is therefore important to increase the amount of light received by a light receiver for increase in measurement accuracy. To this end, the biological information measuring apparatus described in WO 2014/091424 proposes that a reflection film is provided around a light emitter so that a target is allowed to be efficiently irradiated with light emitted from the light emitter.

In the biological information measuring module and the wrist apparatus using the biological information measuring module (biological information measuring apparatus) described in WO 2014/091424, in which the reflection film is provided along the direction in which the light emitted from the light emitter travels toward the target, light traveling in the direction, for example, parallel or perpendicular to the reflection film is not reflected off the reflection film, undesirably resulting in no effect of efficient irradiation of the target with the light emitted from the light emitter.

### SUMMARY

An advantage of some aspects of the invention is to solve at least a part of the problems described above, and the invention can be implemented as the following forms or application examples.

### Application Example 1

A biological information measuring module according to this application example includes a light emitter, a reflector that has a tapered section inclining with respect to the direction in which part of light emitted from the light emitter, light traveling toward a target, and reflects the light, and a light receiver that receives reflected light that is the light reflected off the target.

According to this application example, since the tapered section of the reflector inclines with respect to the traveling direction of the light, light other than the light emitted from the light emitter and traveling toward the target is reflected off the tapered section, and the reflected light is also allowed to travel toward the target. As described above, the light emitted from the light emitter and traveling toward the target and the reflected light that is the light other than the emitted light and reflected off the reflector travel toward the target, whereby the target can be efficiently irradiated with light for measuring biological information.

### Application Example 2

In the biological information measuring module according to the application example described above, it is preferable that a height of the reflector along a direction in which the light exits is smaller than or equal to 770 μm but greater than or equal to 200 μm.

According to this application example, when the height of the reflector is so set as to be smaller than or equal to 770 μm but greater than or equal to 200 μm, the target is allowed to be efficiently irradiated with the light emitted from the light emitter, whereby accurate biological information can be obtained.

Even when the height of the reflector is greater than 770 μm, the distance from the light emitter to an upper portion of the reflector increases, resulting in a decrease in the amount of light reflected off the upper portion of the reflector, and the intensity of the reflected light therefore remains unchanged. On the other hand, the height of the reflector is smaller than 200 μm, the amount of loss of the emitted light increases, and the amount of irradiation necessary for the measurement cannot therefore be obtained, undesirably resulting in a decrease in accuracy of the biological information measurement.

### Application Example 3

In the biological information measuring module according to the application example described above, it is preferable that a height of the reflector is smaller than or equal to 700 μm but greater than or equal to 200 μm.

According to this application example, not only can the target be efficiently irradiated with the light emitted from the light emitter and hence accurate biological information be obtained, but also a thinner biological information measuring module can be achieved.

### Application Example 4

In the biological information measuring module according to the application example described above, it is preferable that a height of the reflector is smaller than or equal to 650 μm but greater than or equal to 200 μm.

According to this application example, not only can the target be efficiently irradiated with the light emitted from the light emitter and hence accurate biological information be obtained, but also a further thinner biological information measuring module can be achieved.

### Application Example 5

In the biological information measuring module according to the application example described above, it is preferable that the reflector has a circular shape in a plan view viewed from the target.

According to this application example, radially spreading light emitted from the light emitter is reflected off the circular tapered section, whereby the target can be efficiently irradiated with the reflected light.

### Application Example 6

In the biological information measuring module according to the application example described above, it is preferable that the reflector has a polygonal shape in the plan view.

According to this application example, the reflector can be efficiently disposed in a space efficient manner, whereby a compact biological information measuring module can be achieved.

### Application Example 7

In the biological information measuring module according to the application example described above, it is preferable that the reflector is higher than the light receiver.

According to this application example, the reflector, which is higher than the light receiver, serves as a light blocking member, whereby a situation in which the light emitted from the light emitter is directly incident on the light receiver as ambient light (noise) can be avoided.

### Application Example 8

In the biological information measuring module according to the application example described above, it is preferable that the reflector is lower than the light receiver.

According to this application example, the thickness of the biological information measuring module can be reduced because the height of the reflector is lowered.

### Application Example 9

In the biological information measuring module according to the application example described above, it is preferable that the light emitter is formed of a plurality of light emitters.

According to this application example, the plurality of light emitters irradiate the target with light having increased intensity, whereby the measurement can be performed with increased accuracy, and accurate biological information can he obtained.

### Application Example 10

In the biological information measuring module according to the application example described above, it is preferable that the plurality of light emitters are arranged in positions symmetric with respect to an imaginary line passing through a center of the light receiver.

According to this application example, when the light emitters are disposed in positions symmetric with respect to an imaginary line passing through the center of the light receiver, the target can be so efficiently irradiated with the light emitted from the plurality of light emitters as to reflect the light toward the light receiver.

### Application Example 11

In the biological information measuring module according to the application example described above, it is preferable that the reflector is provided with a reflection film.

According to this application example, providing the reflector, which reflects the light from the light emitter, with a reflection film contributes to reduction in cost of the reflector.

### Application Example 12

In the biological information measuring module according to the application example described above, it is preferable that the reflector includes a guide section disposed in a position shifted from the tapered section toward the target.

According to this application example, the exiting direction of the light reflected off the tapered section can be determined by the guide section disposed on the side facing the target, whereby the target can be efficiently irradiated with the light.

### Application Example 13

In the biological information measuring module according to the application example described above, it is preferable that in the plan view, a distance between the reflector and the light receiver is smaller than a width of the reflector in a direction in which the reflector and the light receiver are arranged.

According to this application example, the length of the path along which the light is emitted from the light emitter and incident on the target and the light reflected off the target is incident on the light receiver can be reduced, whereby the amount of noise resulting, for example, from entry of ambient light can be reduced, and highly accurate biological information can therefore be obtained.

### Application Example 14

In the biological information measuring module according to the application example described above, it is preferable that a light blocking wall is disposed between the light emitter and the light receiver.

According to this application example, the light blocking wall between the reflector and the light receiver can reliably prevent the light emitted from the light emitter from being directly incident on the light receiver as ambient light (noise).

### Application Example 15

A biological information measuring apparatus according to this application example includes the biological information measuring module according to any of the application examples described above, a first substrate to which a sensor section is connected, and a second substrate to which at least the light emitter, the reflector, and the light receiver contained in the biological information measuring module are connected.

According to this application example, the apparatus can be efficiently assembled by use of two different substrates, the first substrate, to which the sensor section is connected, and the second substrate, to which at least the light emitter, the reflector, and the light receiver are connected.

Further, since the arrangement of the sensor section greatly affects the exterior appearance, in addition to the first substrate, to which the sensor section is connected, the second substrate, to which the light emitter, the reflector, and the light receiver are connected, is provided, whereby the design flexibility and exterior appearance flexibility can be increased.

### Application Example 16

It is preferable that the biological information measuring apparatus according to the application example described above further includes a vibrator that notifies a result of measurement performed by the biological information measuring module in a form of vibration, and the vibrator is preferably disposed in a position where the vibrator does not overlap with the reflector in a plan view viewed from the target,

According to this application example, a situation in which vibration produced by the vibrator directly propagates to the reflector can be avoided, and a situation in which irregular light reflection occurs due to vibration of the reflector and hence the target irradiation efficiency decreases can therefore avoided.

### Application Example 17

It is preferable that the biological information measuring apparatus according to the application example described above further includes a vibrator that notifies a result of measurement performed by the biological information measuring module in a form of vibration, and the vibrator is disposed in a position where the vibrator does not overlap with the light emitter in a plan view viewed from the target.

According to this application example, a situation in which the vibration produced by the vibrator propagates to the light emitter can be avoided. The suppression of the vibration propagation can suppress a decrease in the target irradiation efficiency due to variation in light emission state resulting from vibration of the light emitter.

### Application Example 18

In the biological information measuring apparatus according to the application example described above, it is preferable that the sensor section includes an atmospheric pressure sensor, and that the atmospheric pressure sensor is disposed in a position where the atmospheric pressure sensor does not overlap with the light emitter in a plan view viewed from the target.

According to this application example, the light emitter needs to be so disposed as to face the target, and the atmospheric pressure sensor needs to be provided with a vent for detecting the atmospheric pressure. When the atmospheric pressure sensor and the light emitter are so located in positions where they do not overlap with each other, they are allowed, in their positions, to face the target and a vent can be provided, whereby the thickness of the apparatus can be reduced.

Further, the configuration in which the vent for the atmospheric pressure sensor and the light emitter are separate from each other by a large distance in the plan view can suppress influence on the measurement resulting from a situation in which external light (ambient light) enters the apparatus through the vent and is mixed with the light emitted from the light emitter.

### Application Example 19

In the biological information measuring apparatus according to the application example described above, it is preferable that the sensor section further includes a geomagnetism sensor, and the light emitter is disposed in a position between the geomagnetism sensor and the vibrator in the plan view viewed from the target.

According to this application example, when the light emitter is disposed between the geomagnetism sensor and the vibrator, the distance between the geomagnetism sensor and the vibrator can be increased. Since the geomagnetism sensor tends to be affected by the magnetism emitted from the vibrator, increasing the distance between the geomagnetism sensor and the vibrator allows reduction in influence of the magnetism emitted from the vibrator on the geomagnetism sensor. Therefore, even in the compact biological information measuring apparatus having a restricted size, such as a wrist apparatus as large as a wristwatch, the influence of the magnetic noise on the geomagnetism sensor can be suppressed, whereby the geomagnetism can be stably detected.

### BRIEF DESCRIPTION OF THE DRAWINGS

The invention will be described with reference to the accompanying drawings, wherein like numbers reference like elements.

FIG. 1A is a front-surface-side perspective view of a biological information measuring apparatus according to a first embodiment.

FIG. 1B is a rear-surface-side perspective view of the biological information measuring apparatus according to the first embodiment.

FIG. 2A is a front-surface-side plan view of the biological information measuring apparatus according to the first embodiment.

FIG. 2B is a rear-surface-side plan view of the biological information measuring apparatus according to the first embodiment

FIG. 3 shows the biological information measuring apparatus according to the first embodiment and is a cross-sectional view taken along the line A-A in FIG. 2A.

FIG. 4 is a cross-sectional view showing an example of a biological information measuring module.

FIG. 5 is a plan view showing an example of the arrangement of the biological information measuring module.

FIG. 6A is a plan view showing an example of a detailed configuration of light emitting units.

FIG. 6B is a cross-sectional view showing the example of a detailed configuration of the light emitting units.

FIG. 7 is a cross-sectional view showing another configuration example (variation) of the biological information measuring module.

FIG. 8 is a plan view showing a variation of the light emitting units.

FIG. 9 is a cross-sectional view showing an apparatus body of a biological information measuring apparatus according to a second embodiment

FIG. 10 is a schematic plan arrangement diagram of the biological information measuring apparatus according to the second embodiment

FIG. 11 is a cross-sectional view of a related art example of a biological information measuring apparatus according to a third embodiment.

FIG. 12 is a perspective view showing the biological information measuring apparatus according to the third embodiment.

FIG. 13 is a front view showing a biological information measuring apparatus according to a fourth embodiment.

FIG. 14 is a perspective view showing a biological information measuring apparatus according to a fifth embodiment

FIG. 15 is a cross-sectional view showing a biological information measuring apparatus according to a sixth embodiment.

FIG. 16 is a flowchart showing a method for manufacturing the biological information measuring apparatus according to the third to sixth embodiments.

### DESCRIPTION OF EXEMPLARY EMBODIMENTS

A biological information measuring module according to each embodiment of the invention and a biological information measuring apparatus using the biological information measuring module will be described below. It is not intended that each embodiment described below unduly limits the contents of the invention set forth in the appended claims. Further, all configurations described in each of the embodiments are not necessarily essential configuration requirements of the invention.

### 1. Approach in Each Embodiment of Invention

The approach associated with the biological information measuring module according to each embodiment of the invention and the biological information measuring apparatus using the biological information measuring module will first be described. In a wearable biological information measuring apparatus worn around a user's wrist or any other body site, an approach of acquiring biological information by using a photoelectric sensor is known, as described above. A conceivable biological sensor that is a photoelectric sensor is a biological information measuring module that forms, for example, a pulse wave sensor, and the pulse wave sensor (biological information measuring module) can be used to acquire pulse wave information, such as the pulse rate.

The following description will be made with reference to a wristwatch-shaped apparatus worn around a wrist, but the biological information measuring apparatus according to each embodiment may be worn around the user's any other body site, such as the user's neck and ankle. Further, the biological sensor (biological information measuring module) according to each embodiment of the invention is not limited to a pulse wave sensor and may be a photoelectric sensor that acquires biological information other than the pulse wave information. Moreover, the biological information measuring apparatus according to each embodiment may include a biological sensor other than a photoelectric sensor.

A biological information measuring apparatus including a photoelectric sensor needs to receive necessary light but block unnecessary light. In the case of a pulse wave sensor, since light reflected off a test object under measurement (body site containing blood vessel under measurement, in particular) contains a pulse wave component, intensive reflected light should be received, but the other light, which forms noise components, should be blocked. The term “the other light” conceivably includes direct light that is emitted from a light emitter and directly incident on a light receiver, light reflected off an object other than the test object described above, or environmental light, such as sunlight and illumination light.

### 2. Configuration of Biological Information Measuring Apparatus According to First Embodiment

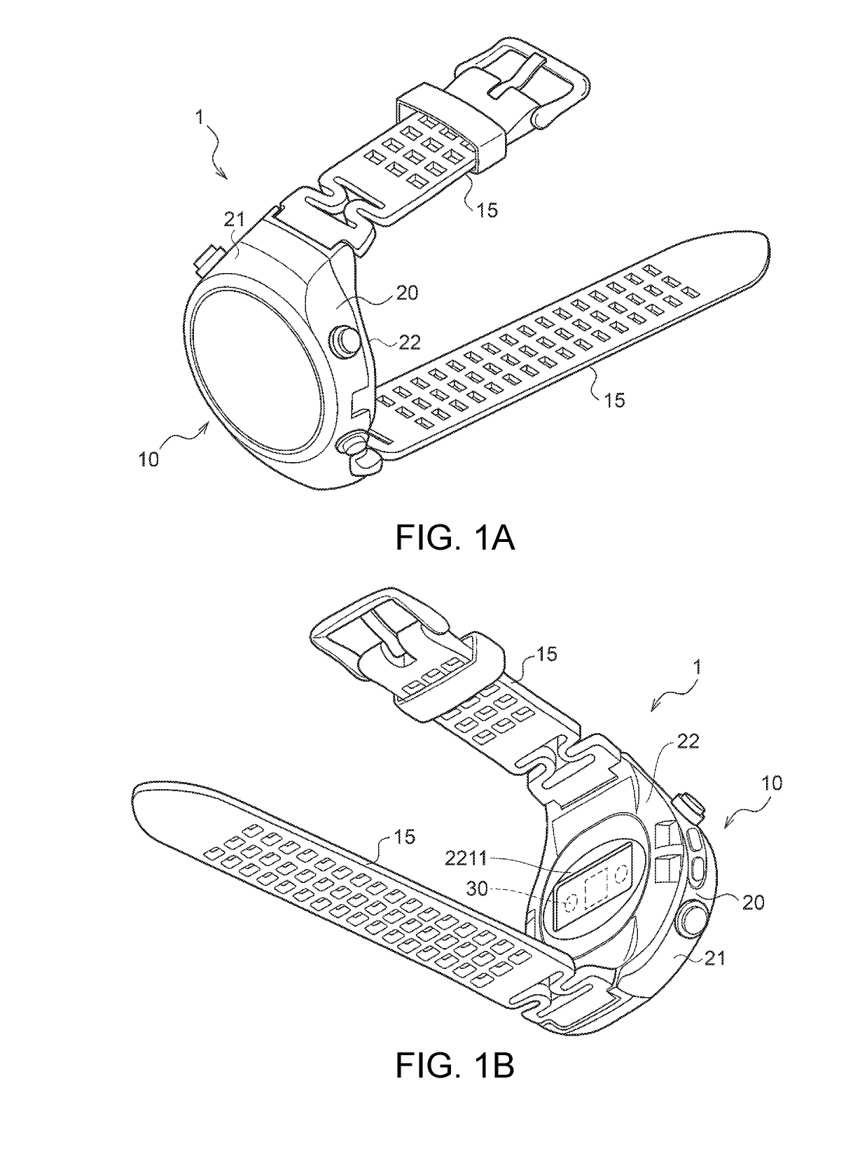

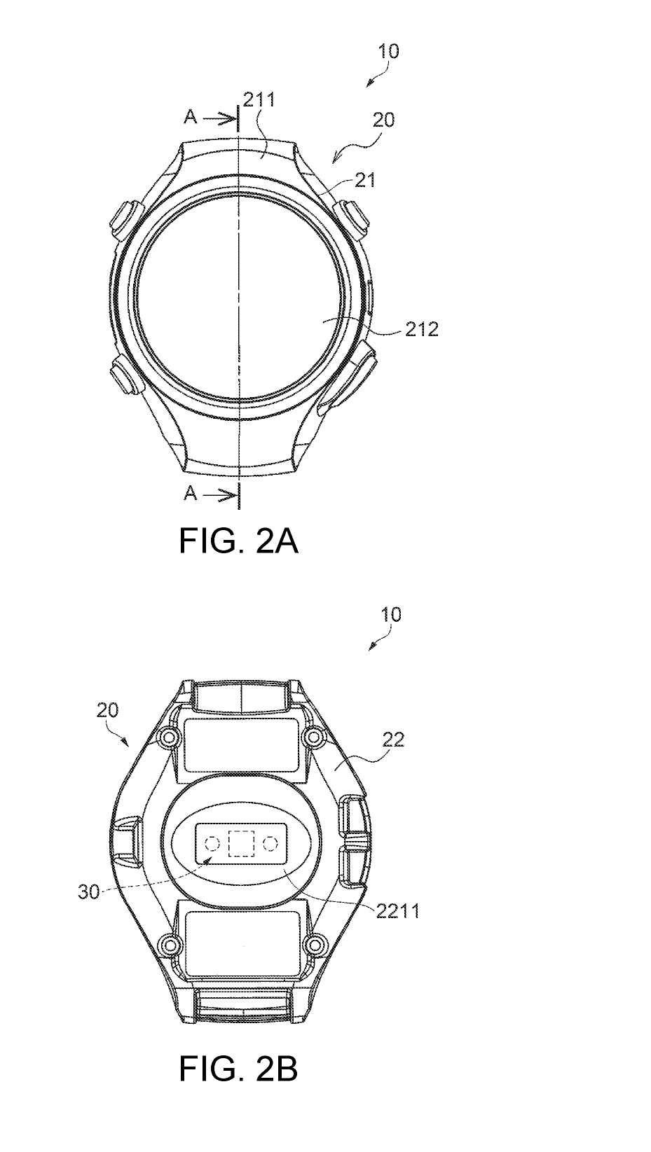

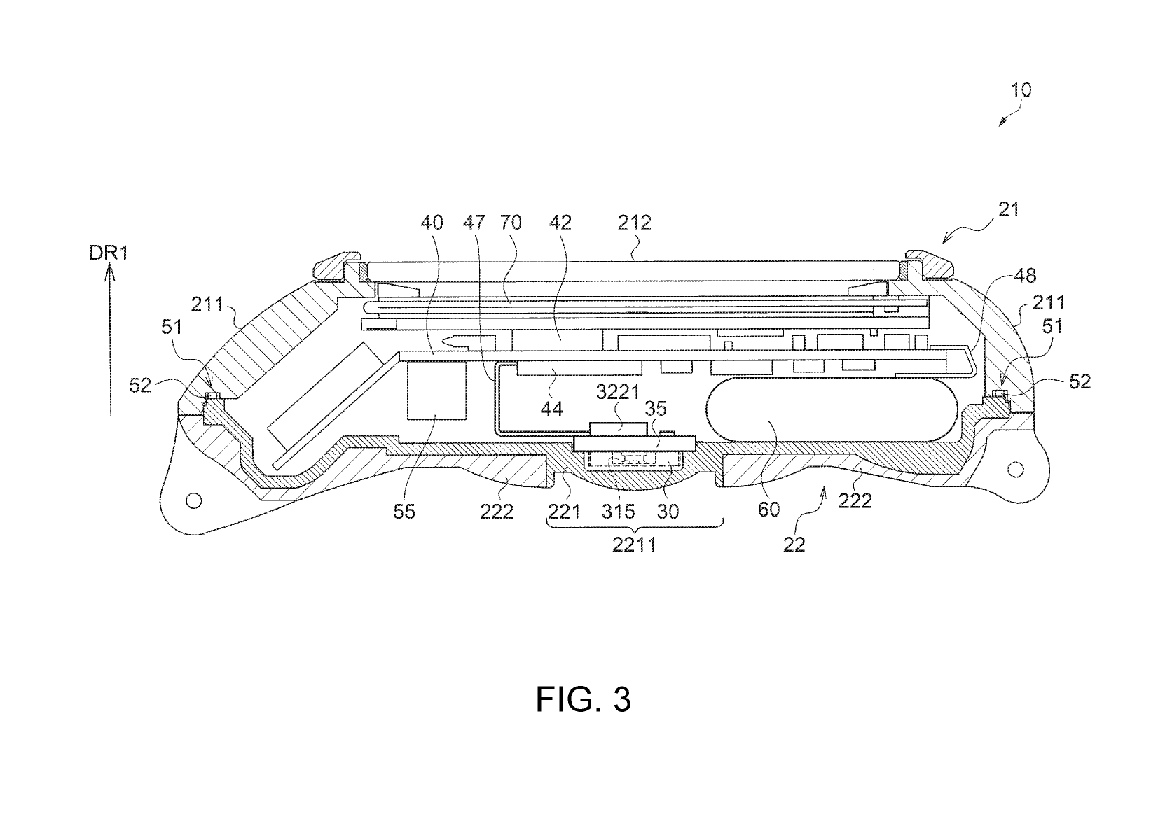

The configuration of a biological information measuring apparatus according to a first embodiment of the invention will next be described with reference to FIGS. 1A, 1B, 2A, 2B, and 3. FIGS. 1A and 1B are perspective views of the biological information measuring apparatus according to the first embodiment. FIG. 1A is a perspective view of the biological information measuring apparatus viewed from the front side thereof, and FIG. 1B is a perspective view of the biological information measuring apparatus viewed from the rear side thereof (target side) opposite the front side. FIG. 2A is a plan view of the biological information measuring apparatus according to the first embodiment viewed from the front side (display surface side). FIG. 2B is a plan view of the biological information measuring apparatus according to the first embodiment viewed from the rear side (target side) opposite the front side. FIG. 3 shows a cross-sectional configuration of the biological information measuring apparatus according to the first embodiment and is a cross-sectional view taken along the line A-A in FIG. 2A.

A biological information measuring apparatus 1 according to the first embodiment is worn by a user at the user's given site (target to be measured, such as wrist) and detects biological information such as pulse wave information. The biological information measuring apparatus 1 includes an apparatus body 10, which comes into intimate contact with the user and detects biological information, and a pair of band sections 15, which are attached to the apparatus body 10 and allow the user to wear the apparatus body 10, as shown in FIGS. 1A and 1B. In the following description, the orientation of the apparatus body 10 worn by the user is defined as follows: The side facing a target to be measured (test object) is called “rear side or rear surface side;” and the display surface side of the apparatus body 10 that is the side opposite the rear side or the rear surface side is called “front side or front surface side.” Further, in the following description, a “target” to be measured is referred to as a “test object” in some cases.

The apparatus body 10 includes a case section 20, which includes a top case 21 and a bottom case 22. The bottom case 22 is located on the side facing the target under measurement when the user wears the apparatus body 10. The top case 21 is disposed on the side (front side) opposite the target under measurement with respect to the bottom case 22. A detection window 2211 is provided in the rear surface of the bottom case 22, and a biological information measuring module 30 is provided in a position corresponding to the detection window 2211.

FIGS. 2A and 2B show the apparatus body 10 of the biological information measuring apparatus 1. Specifically, FIG. 2A is a plan view of the apparatus body 10 viewed in the direction from the top case 21 toward the bottom case 22, and FIG. 2B is a plan view of the apparatus body 10 viewed in the direction opposite the direction in FIG. 2A, that is, in the direction from the bottom case 22 toward the top case 21, that is, in the direction along which the apparatus body 10 is viewed from the side facing the test object (user's wrist) in the situation in which the user wears and use the biological information measuring apparatus 1. That is, FIG. 2A is a plan view primarily showing the structure of the top case 21, and FIG. 2B is a plan view primarily showing the structure of the bottom case 22.

The top case 21 may include a barrel 211 and a glass plate 212, as shown in FIG. 2A. In this case, the barrel 211 and the glass plate 212 may be used as an outer wall that protects the internal structure and may allow the user to view information displayed in a display section, such as a liquid crystal display (hereinafter referred to as LCD 70, see FIG. 3) provided immediately below the glass plate 212, via the glass plate 212, That is, in the biological information measuring apparatus 1 according to the present embodiment, the LCD 70 (see FIG. 3) may be used to display a variety of pieces of information, such as detected biological information, information representing the state of the user's motion, or time information, and present the displayed information to the user via the top case 21. In the above description, a top plate portion of the biological information measuring apparatus 1 is achieved by the glass plate 212 by way of example, and the top plate portion can instead be made of a transparent plastic material or any other non-glass material that forms a transparent member that allows the user to view the LCD 70 and has strength high enough to be capable of protecting the configuration contained in the case section 20, such as the LCD 70.

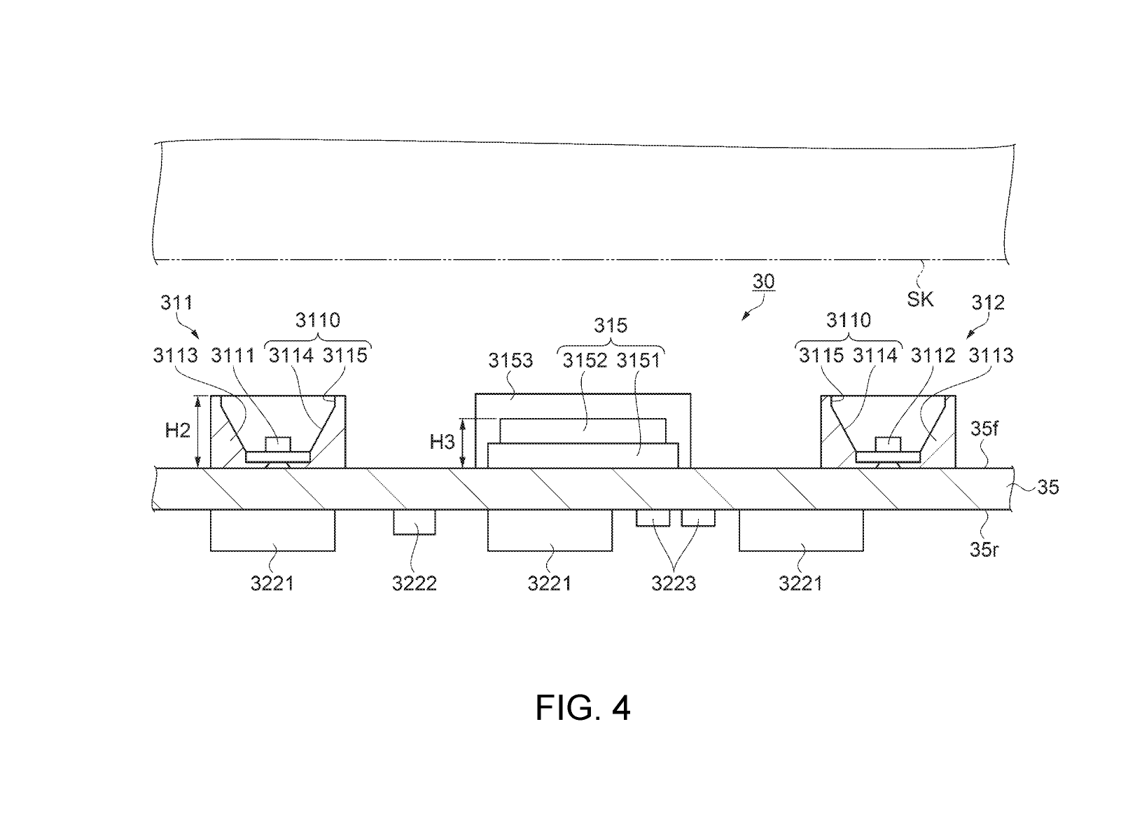

The bottom case 22 is provided with the detection window 2211, and the biological information measuring module 30 is provided in a position corresponding to the detection window 2211, as shown in FIG. 2. The detection window 2211 is configured to transmit light, and light outputted from light emitting units (first light emitting unit 311 and second light emitting unit 312 (see FIG. 4)) contained in the biological information measuring module 30 passes through the detection window 2211 and impinges on the test object (target under measurement). Light reflected off the test object also passes through the detection window 2211 and is received by a light receiver 315 (see FIG. 3) in the biological information measuring module 30. That is, providing the detection window 211 allows biological information detection using a photoelectric sensor. Specifically, the detection window 2211 may be achieved by a light transmissive section 221 (see FIG. 3) (light transmissive section 221 may include detection window 2211). The specific structure of the light transmissive section 221 will be described later.

An example of the detailed cross-sectional structure of the apparatus body 10 of the biological information measuring apparatus 1 will next be described with reference to FIG. 3. FIG. 3 is a cross-sectional view taken along the line A-A in FIG. 2A. In FIG. 3, in the state in which the user (test object) wears the biological information measuring apparatus 1, the direction from the test object toward the case section 20 (direction from bottom case 22 (rear side) toward top case 21 (front side) in narrow sense) is defined as a first direction DR1.

The apparatus body 10 includes, in addition to the top case 21 and the bottom case 22, a module substrate 35 as a second substrate, the biological information measuring module 30, which is connected to the module substrate 35, a circuit substrate 40 as a first substrate, a panel frame 42, a circuit case 44, a geomagnetism sensor 55 as an example of a sensor section, a secondary battery 60, and the LCD 70, as shown in FIG. 3. It however, noted that the configuration of the biological information measuring apparatus 1 is not limited to the configuration shown in FIG. 3; another configuration can be added, and part of the configuration can be omitted. For example, a GPS antenna may be added to the configuration shown in FIG. 3.

The biological information measuring module 30 includes at least the first light emitting unit 311, the second light emitting unit 312, and the light receiver 315, which will be described later with reference to FIG. 4. The first light emitting unit 311 includes a first light emitter 3111 and a reflector 3110, and the second light emitting unit 312 includes a second light emitter 3112 and a reflector 3110. The first light emitting unit 311, the second light emitting unit 312, and the light receiver 315 contained in the biological information measuring module 30 are connected to the module substrate 35 as the second substrate. In other words, the first light emitter 3111, the second light emitter 3112, the reflectors 3110, and the light receiver 315 are connected to the module substrate 35. The module substrate 35 as the second substrate is electrically connected to the circuit substrate 40 as the first substrate, for example, via a flexible substrate 47.

The apparatus body 10 can therefore be efficiently assembled by use of two different substrates, the circuit substrate 40, which serves as the first substrate to which at least the geomagnetism sensor 55 as an example of the sensor section is connected, and the module substrate 35, which serves as the second substrate to which at least the first light emitting unit 311, the second light emitting unit 312, and the light receiver 315 are connected. Further, the design flexibility and exterior appearance flexibility can be increased by use of the two different substrates, although the arrangement of the sensor section greatly affects the exterior appearance.

The biological information measuring apparatus 1 includes, as the biological information measuring module 30, a photoelectric sensor including at least the first light emitting unit 311, the second light emitting unit 312, and the light receiver 315, as shown in FIG. 4, which is the cross-sectional view of the biological information measuring module 30. The biological information measuring apparatus 1 can measure, for example, pulse waves as the biological information by using the characteristics of the photoelectric sensor and derive the pulse rate, hardness of a blood vessel, the user's motion state, the user's mental state, and other factors on the basis of the measured pulse waves.

In the photoelectric sensor, a light collection mirror (not shown) collects light radiated from the light emitters (first light emitter 3111 and second light emitter 3112) such as LEDs (light emitting diodes) toward a test object SK (user's wrist, for example) shown in FIG. 4 and reflected off a blood vessel in the wrist, and the light receiver 315 such as a photodiode receives the reflected light. In this process, the photoelectric sensor measures the user's pulse by using a phenomenon in which light reflectance of an expanding blood vessel differs from the light reflectance of a contracting blood vessel. The biological information measuring module 30 is therefore preferably pressed against the wrist, more preferably in intimate contact with the wrist so that the light receiver 315 in the photoelectric sensor receives no light that forms measurement noise. An example of the specific configuration of the biological information measuring module 30 will be described later with reference to FIG. 4 and other figures.

The circuit substrate 40 as the first substrate has the panel frame 42, which guides the LCD 70 or any other display panel, disposed on one surface of the circuit substrate 40 and further has the circuit case 44, which guides the secondary battery 60 and other components, disposed on the other surface of the circuit substrate 40, as shown in FIG. 3. The circuit substrate 40 is formed, for example, of a substrate made of an epoxy-based material containing glass fibers, and a wiring pattern formed, for example, of a copper foil is formed on each of the surfaces of the circuit substrate 40. The panel frame 42 and the circuit case 44 are preferably made of polyacetal, polycarbonate, or any other resin.

On the circuit substrate 40 are mounted elements that form a circuit that drives the photoelectric sensor (biological information measuring module 30) to cause it to measure the pulse, a circuit that drives the LCD 70, a circuit that controls the circuits described above, and other circuits. Electrodes to be connected to the LCD 70 are formed on the one surface of the circuit substrate 40 and electrically continuous with electrodes of the LCD 70 via a connector that is not shown. The LCD 70 displays measured pulse data such as the pulse rate, time information such as the current time, and other pieces of information in accordance with each mode of the biological information measuring apparatus 1.

The circuit case 44 accommodates the secondary battery 60 (lithium secondary battery), which is a rechargeable battery. The positive and negative terminals of the secondary battery 60 are connected to the circuit substrate 40, for example, via a connection substrate 48, and the secondary battery 60 supplies electric power to a circuit that controls the electric power. The electric power is converted into predetermined voltage or otherwise processed by the circuit and supplied to each of the circuits described above to operate the circuit that drives the biological information measuring module 30 to cause it to measure the pulse, the circuit that drives the LCD 70, the circuit that controls the circuits described above, and other circuits. The secondary battery 60 is charged via a pair of charge terminals electrically continuous with the circuit substrate 40 via an electrically conducting member (not shown) such as a coil spring. In the above description, the secondary battery 60 is used as a battery by way of example, and the battery may instead be a primary battery, which does not need to be charged.

The cross-sectional structure of each of the light transmissive section 221 and a light blocking section 222 will next be described in detail. The light blocking section 222 is so provided in the portion excluding the detection window 2211 as to cover the light transmissive section 221 on the side facing the test object, as shown in FIG. 3.

The light transmissive section 221 is not covered with the light blocking section 222 in the detection window 2211. In other words, the detection window 2211 is achieved by the light transmissive section 221. Therefore, in the photoelectric sensor provided in the biological information measuring module 30, the test object as the target under measurement can be irradiated with light from the first light emitting unit 311 and the second light emitting unit 312, and the light receiver 315 can receive light reflected off the test object, whereby biological information such as pulse wave information can be detected, as described above.

On the other hand, in the portion excluding the detection window 2211, the light transmissive section 221 is covered with the light blocking section 222 on the side facing the test object (side facing away from first direction DR1 shown in FIG. 3). The configuration described above can restrict entry of light into the photoelectric sensor provided in the biological information measuring module 30. Therefore, light that is desired to be received, that is, light radiated from the first light emitting unit 311 and the second light emitting unit 312 and reflected off the test object can be received, and light that serves as a noise source, for example, environmental light such as sunlight and illumination light, can be blocked at the same time, whereby biological information can be detected with improved accuracy.

The structure in which the light blocking section 222 covers the light transmissive section 221 can be grasped from another viewpoint. Specifically, in the portion excluding the detection window 2211, the light transmissive section 221 is provided on one side of the light blocking section 222, the side thereof facing in the first direction DR1. Since the light transmissive section 221 transmits light, a portion where the light transmissive section 221 is provided needs to be configured in consideration of possibility of entry of light through the portion. Since the light transmissive section 221 is provided on the bottom case 22, the light incident direction that should be taken into consideration is the direction from the test object toward the bottom case 22, that is, the first direction DR1. In this case, when the light transmissive section 221 is provided on the DR1-side of the light blocking section 222, light traveling toward the light transmissive section 221 in the portion excluding the detection window 2211 is believed to be blocked by the light blocking section 222, whereby entry of light that serves as a noise source into the biological information measuring module 30 can be avoided.

It is noted that providing the light transmissive section 221 on the DR1-side of the light blocking section 222 does not mean that the light transmissive section 221 is provided on the DR1-side of the entire light blocking section 222, as seen from the example shown in FIG. 3. For example, the light transmissive section 221 may not be disposed on part of the DR1-side of the light blocking section 222, such as an area around the biological information measuring module 30. That is, providing the light transmissive section 221 on the DR1-side of the light blocking section 222 may be providing the light blocking section 222 on one side of the provided light transmission section 221, the side thereof opposite the DR1 direction, except the detection window 2211.

To put the configuration described above in other words, in the biological information measuring apparatus 1 according to the present embodiment, the light blocking section 222 is so provided as to overlap with the light transmissive section 221 on the test-object-side of the light transmissive section 221 in the portion except the detection window 2211. That is, in the portion where the light blocking section 222 overlaps with the light transmissive section 221 on the test object side, the light blocking section 222 blocks light traveling from the exterior of the case section 20 toward the interior thereof, whereas in the portion where the light blocking section 222 does not overlap with the light transmissive section 221, the light enters the case section 20 (biological information measuring module 30 in narrow sense). Therefore, the light is allowed to pass through the detection window 2211, but the light can be blocked by the portion excluding the detection window 2211, as described above.

The light transmissive section 221 is made of a resin material, and the light blocking section 222 is made of a glass-containing resin material, which is a resin material with which glass (glass fibers in narrow sense) is impregnated. Specifically, the light transmissive section 221 contains any of polycarbonate, an ABS resin, or an acrylic resin, and the light blocking section 222 contains any of glass-containing polycarbonate, a glass-containing ABS resin, or a glass-containing acrylic resin.

That is, the light blocking section 222 according to the present embodiment may be made of an FRP (fiber reinforced plastic) material, particularly, a GFRP (glass fiber reinforced plastic) material using glass fibers as the fibers for reinforcement. As the resin used along with the glass fibers in a GFRP material, a thermoplastic resin may be used. In the present embodiment, polycarbonate or an ABS resin can be used as the thermoplastic resin, It is known that acrylic resins are classified into thermoplastic resins and thermosetting resins. In the present embodiment, either of the two types of acrylic resin can be used. Since GFRP materials are relatively inexpensive and typical among FRP materials, the light blocking section 222 according to the present embodiment can be readily achieved by use of a GFRP material. As the resin material in a GFRP material, a polyester resin, a vinyl ester resin, an epoxy resin, a phenol resin, or a variety of other resin materials can be used, and the light blocking section 222 according to the present embodiment can be made of any of the wide variety of materials described above For example, a resin material with which glass fibers are impregnated is not limited to a resin material made only of polycarbonate, an ABS resin, or an acrylic resin and may be an alloy material that is a combination thereof as a variation.

The light transmissive section 221 may be so formed as to extend from the detection window 2211 to a sealing section 51, which is provided at the portion where the top case 21 and the bottom case 22 are connected to each other. The sealing section 51 may be provided with a gasket 52, which hermetically closes the interior of the case section 20 against the exterior thereof. The gasket 52 is provided in the portion where the top case 21 and the bottom case 22 are connected to each other and hermetically closes the interior of the case section 20 against the exterior thereof.

### 3. Example of Configuration of Biological Information Measuring Module

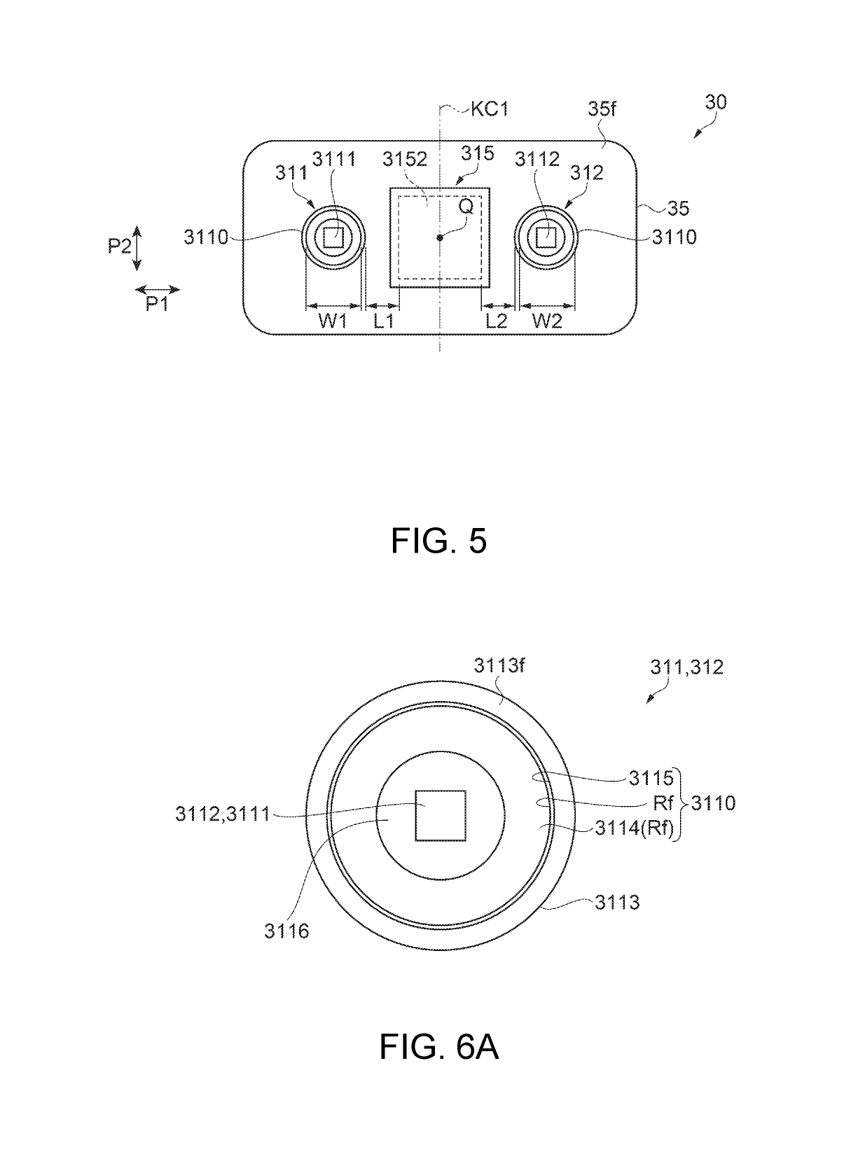

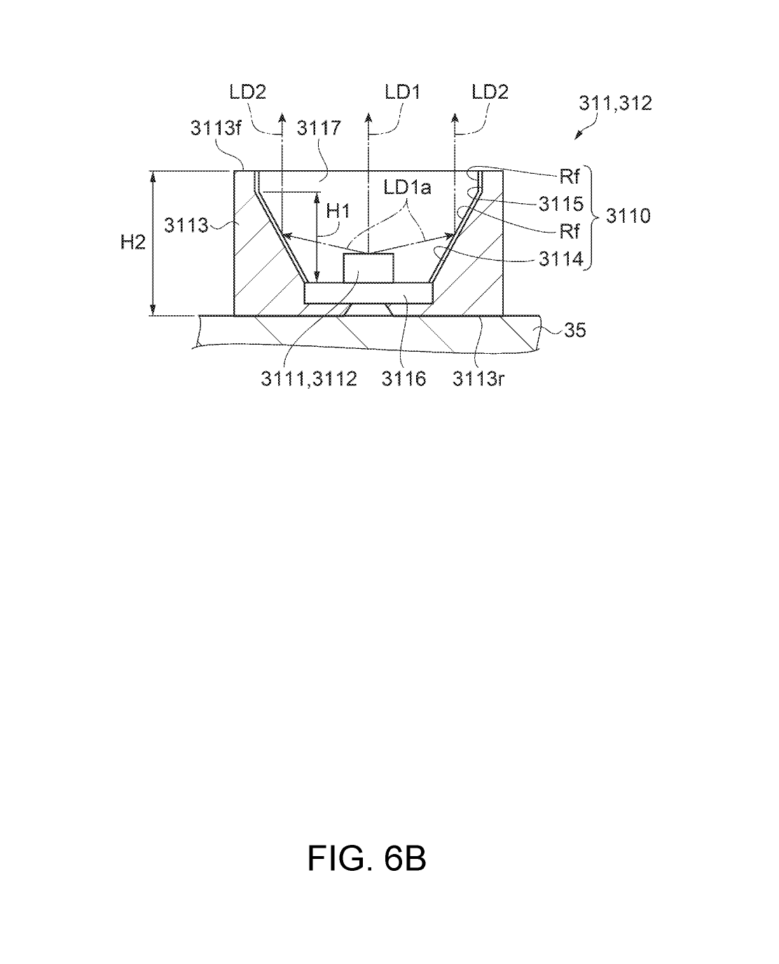

An example of the configuration of the biological information measuring module 30 according to the present embodiment will be described with reference to FIGS. 4, 5, 6A, and 6B. FIG. 4 is a cross-sectional view showing an example of the biological information measuring module. FIG. 5 is a plan view showing an example of the arrangement of the biological information measuring module. FIG. 6A is a plan view showing an example of a detailed configuration of the light emitting units. FIG. 6B is a cross-sectional view showing the example of a detailed configuration of the light emitting units. In FIGS. 4, 5, 6A, and 6B, the configuration of the biological information measuring module and the configurations of the first light emitting unit 311 and the second light emitting unit 312 according to the present embodiment are diagrammatically shown for simplification of the illustration, and the dimensions and ratios in the figures differ from actual values.

The biological information measuring apparatus 1 includes the biological information measuring module 30, as described above. The biological information measuring module 30 includes the photoelectric sensor including at least the plurality of light emitting units (first light emitting unit 311 and second light emitting unit 312) and the light receiver 315, as shown in FIGS. 4 and 5. In other words, the biological information measuring apparatus 1 includes, as the biological information measuring module 30, the photoelectric sensor including at least the plurality of light emitters (first light emitter 3111 and second light emitter 3112), the reflectors 3110, and the light receiver 315. The biological information measuring module 30 may further include first circuit parts 3221, which drive the photoelectric sensor, other circuit parts 3222 and 3223, and other parts. The first circuit parts 3221, the other circuit parts 3222 and 3223, and other components can be connected to the other surface 35r of the module substrate 35.

The first light emitting unit 311 and the second light emitting unit 312 include the respective light emitters (first light emitter 3111 and second light emitter 3112) and reflectors 3110. The reflectors 3110 each include a body section 3113, which includes a tapered section 3114, which surrounds the first light emitter 3111 and the second light emitter 3112 circularly in a plan view, and a guide section 3115, which is disposed in a position shifted from the tapered section 3114 toward the test object SK (target). The first light emitting unit 311 and the second light emitting unit 312 emit light toward the test object SK.

The light receiver 315 includes a light receiving device 3152 such as a photodiode, which is connected and fixed onto a die pad 3151, and a molding resin 3153, which has translucency and coats at least the outer circumference of the light receiving device 3152. The light receiver 315 can receive light emitted from the first light emitter 3111 and the second light emitter 3112 and reflected off the test object SK.

The first light emitting unit 311 (first light emitter 3111 and reflector 3110), the second light emitting unit 312 (second light emitter 3112 and reflector 3110), and the light receiver 315 are connected to one surface 35f of the module substrate 35 as the second substrate. Specifically, the first light emitter 3111 and the second light emitter 3112 are located on opposite sides of the light receiver 315, and the first light emitter 3111, the second light emitter 3112, and the light receiver 315 are arranged in a single row. The first light emitter 3111 and the second light emitter 3112 are preferably disposed in positions symmetric with respect to an imaginary line KC1, which passes through the center Q of the light receiver 315 in a plan view of the module substrate 35 (plan view viewed from side facing test object SK) as shown in FIG. 5.

Since the first light emitter 3111 (first light emitting unit 311) and the second light emitter 3112 (second light emitting unit 312), which are the plurality of light emitters, and the light receiver 315 are arranged as described above, the test object SK is irradiated with light having increased intensity, whereby the measurement can be performed with increased accuracy, and accurate biological information can be obtained.

Further, the light outputted from each of the plurality of light emitters (first light emitter 3111 and second light emitter 3112) spreads in a radial pattern. Therefore, when the first light emitter 3111 and the second light emitter 3112 are disposed in positions symmetric with respect to the imaginary line KC1, which passes through the center Q of the light receiver 315, the test object SK can be so irradiated with the light emitted from the plurality of light emitters (first light emitter 3111 and second light emitter 3112) as to efficiently reflect the light toward the light receiver 315.

Further, in the plane view viewed from the side facing the test object SK, the distances L1 and L2 between the reflectors 3110 and the light receiver 315 are preferably smaller than the widths W1 and W2 of the reflectors 3110 in the direction in which the reflectors 3110 and the light receiver 315 are arranged (direction along P1 in FIG. 5). Specifically, the distance L1 between the reflector 3110 on the side where the first light emitter 3111 is present and the light receiver 315 is smaller than the width W1 of the reflector 3110 on the side where the first light emitter 3111 is present, and the distance L2 between the reflector 3110 on the side where the second light emitter 3112 is present and the light receiver 315 is smaller than the width W2 of the reflector 3110 on the side where the second light emitter 3112 is present.

The arrangement described above allows reduction in the length of the path along which the light is emitted from the light emitters (first light emitter 3111 and second light emitter 3112) and incident on the test object SK and the light reflected off the test object SK is incident the light receiver 315, whereby the amount of noise resulting, for example, from entry of ambient light can be reduced, and highly accurate biological information can therefore be obtained.

The first light emitting unit 311 and the second light emitting unit 312 will be described below in detail with reference to FIGS. 6A and 6B. The first light emitting unit 311 and the second light emitting unit 312 include the respective light emitters (first light emitter 3111 and second light emitter 3112) and reflectors 3110, as shown in FIGS. 6A and 6B. Specifically, the first light emitting unit 311 includes the first light emitter 3111 and the reflector 3110. The second light emitting unit 312 includes the second light emitter 3112 and the reflector 3110.

The first light emitter 3111 and the second light emitter 3112 are each formed, for example, of an LED (light emitting diode). The first light emitter 3111 and the second light emitter 3112 are each connected to a die pad 3116 fixed to a lower portion (portion facing module substrate 35) of the body section 3113 of the corresponding reflector 3110, which will be described later.

The reflectors 3110 each include the body section 3113, which includes the tapered section 3114, which surrounds the first light emitter 3111 and the second light emitter 3112 circularly in a plan view, and the guide section 3115, which disposed in a position shifted from the tapered section 3114 toward the test object SK (target). The tapered section 3114 and the guide section 3115 are provided on the body section 3113, specifically, the inner wall surface thereof facing a hollow space 3117 (see FIG. 6B) formed in a central portion of the body section 3113. The tapered section 3114 inclines, in such a way that the hollow space 3117 widens, along the traveling direction of light LD1 emitted from the first light emitter 3111 and the second light emitter 3112 and travels toward the test object SK.

The reflectors 3110 each preferably have circular outer and inner circumferences in the plan view viewed from the test object SK, as shown in FIG. 6A. When the reflectors 3110 each have a circular shape in the plan view as described above, the radially spreading light emitted from the first light emitter 3111 and the second light emitter 3112 is reflected off the circular tapered sections 3114, whereby the test object SK can be efficiently irradiated with the reflected light.

The tapered sections 3114 are each preferably provided with a reflection film Rf capable of reflecting, as light LD2, light LD1a emitted from the first light emitter 3111 and the second light emitter 3112. The reflection film Rf may be provided on each of the guide sections 3115. The reflection film Rf can be readily formed at low cost and can therefore contribute to reduction in cost of the reflectors 3110.

The reflectors 3110 are each preferably so configured that the height H1 from the module substrate 35 to the upper end of the tapered section 3114 in the direction along the exiting direction of the light LD1 emitted from the first light emitter 3111 and the second light emitter 3112 is smaller than or equal to 770 μm but greater than or equal to 200 μm, as shown in FIG. 6B. The height H1 to the upper end of the tapered section 3114 is in detail the distance from the point where the tapered section 3114 is connected to the guide section 3115 (upper end of tapered section 3114) to the one surface 35f, which is a surface of the module substrate 35 and to which the reflectors are connected.

When the height H1 of the tapered sections 3114 is smaller than or equal to 770 μm but greater than or equal to 200 μm, the test object SK can be efficiently irradiated with the light emitted from the first light emitter 3111 and the second light emitter 3112, whereby accurate biological information can be obtained.

When the height of the reflectors 3110, specifically, the height H1 of the tapered sections 3114 is greater than 770 μm, the distance from the first light emitter 3111 and the second light emitter 3112 to upper portions of the tapered sections 3114 increases, resulting in a decrease in the amount of light reflected off the upper portions of the tapered sections 3114, and the intensity of the reflected light therefore substantially remains unchanged. On the other hand, when the height H1 of the tapered sections 3114 is smaller than 200 μm, the amount of light that does not impinge on the tapered sections 3114 but passes by the tapered sections 3114 increases. That is, the amount of loss of the light emitted from the first light emitter 3111 and the second light emitter 3112 increases, and the amount of irradiation necessary for the measurement cannot therefore be obtained, undesirably resulting in a decrease in accuracy of the biological information measurement.

The reflectors 3110 are each more preferably so configured that the height H1 from the module substrate 35 to the upper end of the tapered section 3114 in the direction along the exiting direction of the light LD1 emitted from the first light emitter 3111 and the second light emitter 3112 is smaller than or equal to 700 μm but greater than or equal to 200 μm.

When the height H1 of the tapered sections 3114 is smaller than or equal to 700 μm but greater than or equal to 200 μm as described above, not only can the test object SK be efficiently irradiated with the light emitted from the first light emitter 3111 and the second light emitter 3112 and hence accurate biological information be obtained, but also the height of the reflectors 3110 can be reduced and a thinner biological information measuring module 30 can therefore be achieved.

The reflectors 3110 are each more preferably so configured that the height H1 from the module substrate 35 to the upper end of the tapered section 3114 in the direction along the exiting direction of the light LD1 emitted from the first light emitter 3111 and the second light emitter 3112 is smaller than or equal to 650 μm but greater than or equal to 200 μm.

When the height H1 of the tapered sections 3114 is smaller than or equal to 650 μm but greater than or equal to 200 μm as described above, not only can the test object SK be efficiently irradiated with the light emitted from the first light emitter 3111 and the second light emitter 3112 and hence accurate biological information be obtained but also the height of the reflectors 3110 can be reduced and a particularly thin biological information measuring module 30 can therefore be achieved.

The reflectors 3110 are each preferably further so configured that the height H2 from the module substrate 35 to the upper surface 3113f of the guide section 3115 in the direction along the exiting direction of the light LD1 emitted from the first light emitter 3111 and the second light emitter 3112 is greater than the height H3 of the light receiver 315 (see FIG. 4).

When the height H2 of the reflectors 3110 is greater than the height H3 of the light receiver 315 as described above, the reflectors 3110, which are higher than the light receiver 315, serve as light blocking members, whereby a situation in which the light emitted from the first light emitter 3111 and the second light emitter 3112 is directly incident on the light receiver 315 as ambient light (noise) can be avoided.

The reflectors 3110 can each be instead so configured that the height H2 from the module substrate 35 to the upper surface 3113f of the guide section 3115 in the direction along the exiting direction of the light LD1 emitted from the first light emitter 3111 and the second light emitter 3112 is smaller than the height H3 of the light receiver 315 (see FIG. 4). The height H2 from the module substrate 35 to the upper surface 3113f of each of the guide sections 3115 can be, in other words, the height of the reflectors 3110.

When the height H2 of the reflectors 3110 is smaller than the height H3 of the light receiver 315 as described above, the height of the reflectors 3110 can be reduced, and the thickness of the biological information measuring module 30 can therefore be reduced.

According to the biological information measuring module 30 according to the first embodiment described above and the biological information measuring apparatus 1 using the biological information measuring module 30, at least the following advantageous effects can be provided.

According to the biological information measuring module 30, since the tapered sections 3114 of the reflectors 3110 incline with respect to the traveling direction of the light LD1, light other than the light emitted from the light emitters (first light emitter 3111 and the second light emitter 3112) and traveling toward the test object SK (target) is reflected off the tapered sections 3114, and the reflected light also travels toward the test object SK. As described above, the light emitted from the light emitters (first light emitter 3111 and the second light emitter 3112) and traveling toward the test object SK (target) and the reflected light that is the light other than the emitted light and reflected off the reflectors 3110 (tapered sections 3114) travel toward the test object SK, whereby the test object SK can be efficiently irradiated with light for measuring biological information.

Further, the biological information measuring apparatus 1 using the biological information measuring module 30 uses two different substrates, the circuit substrate 40 as the first substrate to which the geomagnetism sensor 55 disposed as an example of the sensor section is connected, and the module substrate 35 as the second substrate to which at least the first light emitting unit 311, the second light emitting unit 312, and the light receiver 315 are connected. The configuration in which the circuit substrate 40, which forms the sensor section, and the module substrate 35, which forms the biological information measuring module 30, are individual substrates allows efficient assembly of the constituent parts of the biological information measuring apparatus 1. Further, the design flexibility and exterior appearance flexibility of the biological information measuring apparatus 1 can be increased by use of the two different substrates, although the arrangement of the sensor section greatly affects the exterior appearance.

### 4. Variation of Biological Information Measuring Module

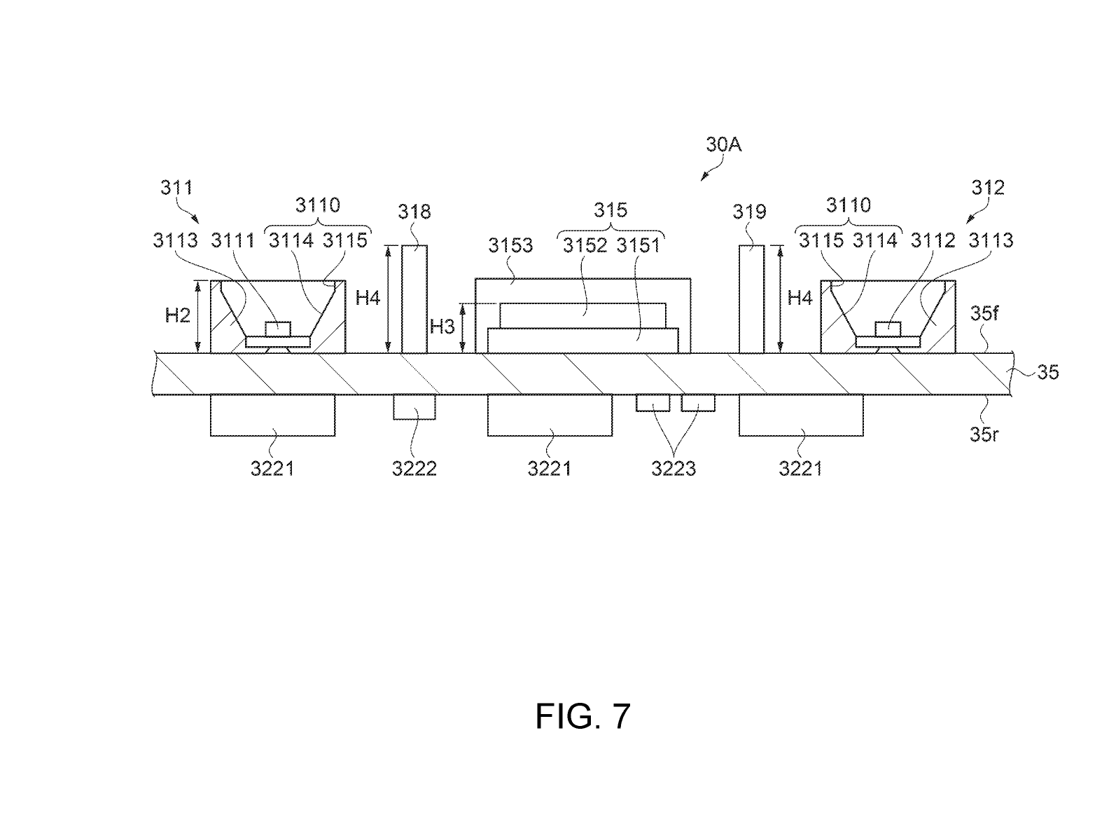

As the biological information measuring module 30, another configuration example (variation) shown below is applicable. FIG. 7 is a cross-sectional view showing another configuration example (variation) of the biological information measuring module. In FIG. 7, the configuration of the biological information measuring module according to the present variation is diagrammatically shown for simplification of the illustration, and the dimensions and ratios in FIG. 7 differ from actual values. Further, in the following description, the same configurations as those in the first embodiment described above have the same reference characters and will not be described in some cases.

A biological information measuring module 30A according to the other configuration example (variation) includes a photoelectric sensor including at least the plurality of light emitting units (first light emitting unit 311 and second light emitting unit 312) and the light receiver 315 having the same configurations as those in the first embodiment, as shown in FIG. 7. The first light emitting unit 311 includes the first light emitter 3111 and the reflector 3110, and the second light emitting unit 312 includes the second light emitter 3112 and the reflector 3110. The biological information measuring module 30A further includes light blocking walls (first light blocking wall 318 and second light blocking wall 319) that prevent the light from the first light emitting unit 311 and the second light emitting unit 312 from directly entering the light receiver 315. The biological information measuring module 30A further includes the first circuit parts 3221, which drive the photoelectric sensor, the other circuit parts 3222 and 3223, and other components, as in the first embodiment.

The first light emitting unit 311 (first light emitter 3111 and reflector 3110), the second light emitting unit 312 (second light emitter 3112 and reflector 3110), and the light receiver 315 are connected to the one surface 35f of the module substrate 35 as the second substrate. Specifically, the first light emitter 3111 and the second light emitter 3112 are located on opposite sides of the light receiver 315, and the first light emitter 3111, the second light emitter 3112, and the light receiver 315 are arranged in a single row.

The first light blocking wall 318 is disposed between the first light emitting unit 311 and the light receiver 315, and the second light blocking wall 319 is disposed between the second light emitting unit 312 and the light receiver 315. The light blocking walls (first light blocking wall 318 and second light blocking wall 319) are made of a material that transmits no light and formed in a plate-like shape having wall surfaces (front and rear surfaces) facing the first light emitting unit 311/second light emitting unit 312 and the light receiver 315. The height H4 of the light blocking walls (first light blocking wall 318 and second light blocking wall 319) from the one surface 35f of the module substrate 35 is preferably so set as to be greater than the height H2 of the first light emitter 3111 and the second light emitter 3112 (reflectors 3110) from the one surface 35f of the module substrate 35 and the height H3 of the light receiver 315 from the one surface 35f of the module substrate 35.

When the thus configured light blocking walls (first light blocking wall 318 and second light blocking wall 319) are provided between the first light emitting unit 311 (first light emitter 3111 and reflector 3110) and the light receiver 315 and between the second light emitting unit 312 (second light emitter 3112 and reflector 3110) and the light receiver 315, a situation in which the light emitted from the first light emitter 3111 and the second light emitter 3112 is directly incident as ambient light (noise) on the light receiver 315 can be reliably avoided.

### 5. Variation of Light Emitting Units

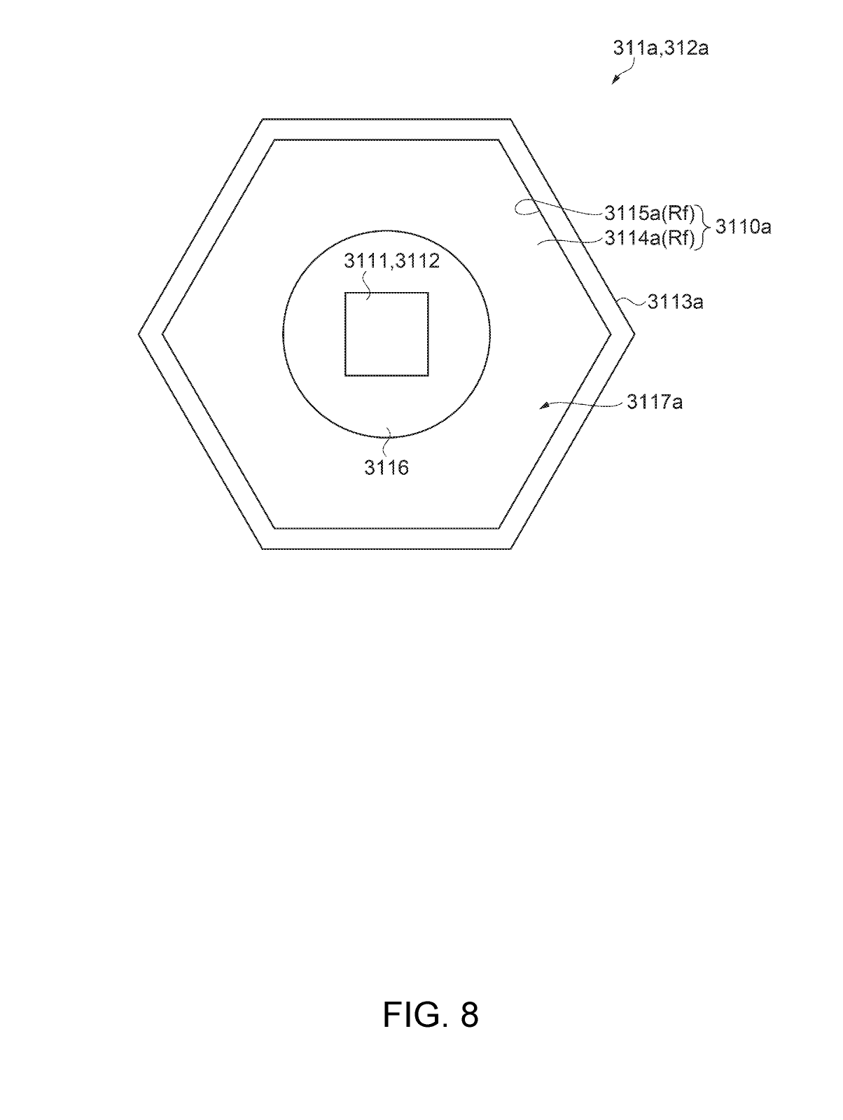

A variation of the light emitting units (first light emitting unit and second light emitting unit) will next be described with reference to FIG. 8. FIG. 8 is a plan view showing a variation of the light emitting units (first light emitting unit and second light emitting unit). In FIG. 8, the configuration of the light emitting units according to the present variation is diagrammatically shown for simplification of the illustration, and the dimensions and ratios in FIG. 8 differ from actual values. Further, in the following description, the same configurations as those in the first embodiment described above have the same reference characters and will not be described in some cases.

The light emitting units (first light emitting unit 311a and second light emitting unit 312a) according to the variation shown in FIG. 8 each include a light emitter (first light emitter 3111 or second light emitter 3112) and a reflector 3110a. The reflectors 3110a each include a body section 3113a, which includes a tapered section 3114a, which has a polygonal shape in a plan view, a hexagonal shape in the present example, and surrounds the first light emitter 3111 and the second light emitter 3112, and a guide section 3115a, which is disposed in a position shifted from the tapered section 3114a toward a test object (target). The body section 3113a is also allowed to have an outer circumferential shape that conforms to the polygonal shapes of the tapered section 3114a and the guide section 3115a (hexagonal shapes in present example).

The tapered section 3114a and the guide section 3115a are provided on the body section 3113a, specifically, the inner wall surface thereof facing a hollow space 3117a formed in a central portion of the body section 3113a. The tapered section 3114a inclines, in such a way that the hollow space 3117a widens, along the traveling direction of the light LD1 emitted from the first light emitter 3111 and the second light emitter 3112 and travels toward the test object. The tapered section 3114a and the guide section 3115a are provided with the reflection film Rf capable of reflecting the light LD1a emitted from the first light emitter 3111 and the second light emitter 3112.

According to the light emitting units (first light emitting unit 311a and second light emitting unit 312a) according to the variation, in which the reflectors 3110a have a polygonal shape (hexagonal shape in present example) in a plan view, the reflectors 3110a can be efficiently disposed in a space efficient manner, whereby a compact biological information measuring module 30 can be achieved.

### 6. Configuration of Biological Information Measuring Apparatus According to Second Embodiment

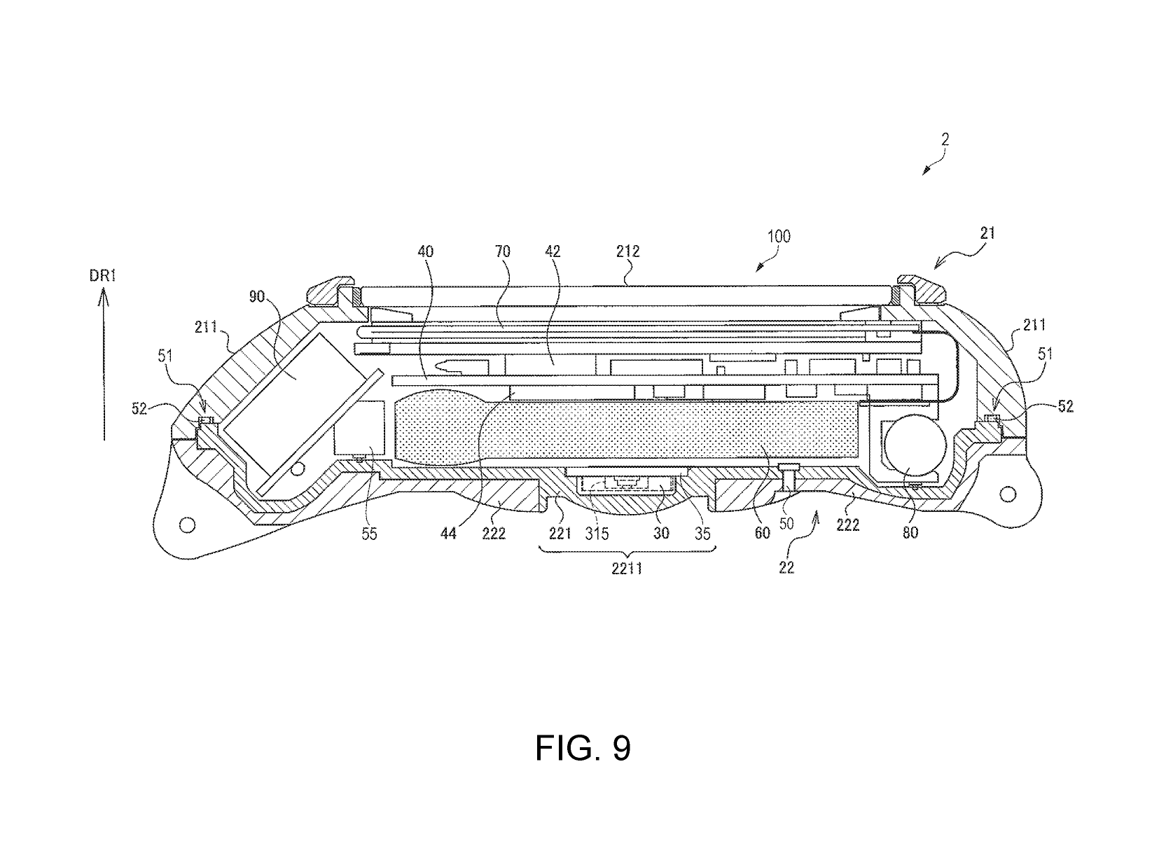

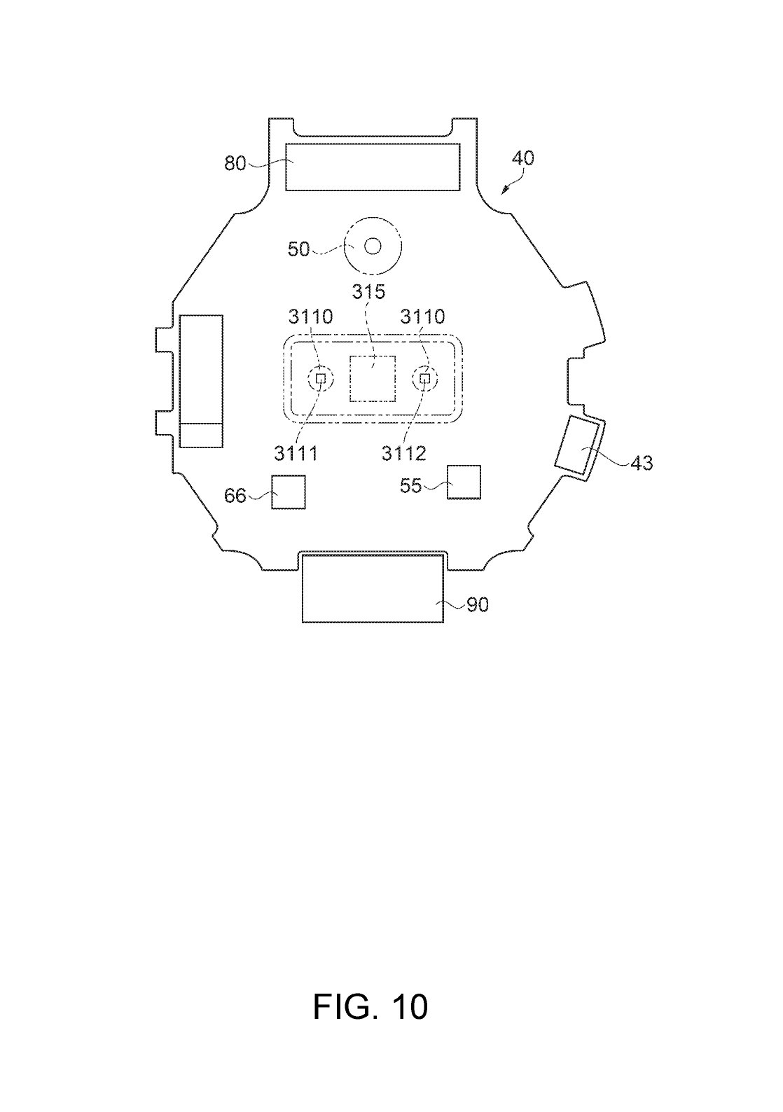

The configuration of a biological information measuring apparatus according to a second embodiment of the invention will next be described with reference to FIGS. 9 and 10. FIG. 9 is a cross-sectional view showing an apparatus body of the biological information measuring apparatus according to the second embodiment. FIG. 10 is a schematic plan arrangement diagram of the biological information measuring apparatus according to the second embodiment. In the following description, the same configurations as those in the first embodiment described above have the same reference characters and will not be described in some cases. Further, in the following description, the orientation of an apparatus body 100 worn by the user is defined as follows: The side facing a target to be measured is called “rear side or rear surface side;” and the side facing the front surface of the apparatus body 100 and opposite the rear side or the rear surface side is called “front side or front surface side.”

A biological information measuring apparatus 2 according to the second embodiment is worn by a user at the user's given site (such as wrist) and detects biological information such as pulse wave information. The biological information measuring apparatus 2 includes the apparatus body 100, which comes into intimate contact with the user and detects biological information, and a band section (not shown) that is attached to the apparatus body 100 and allows the user to wear the apparatus body 100.

The apparatus body 100 shown in FIG. 9 includes the top case 21 and the bottom case 22, as in the first embodiment. The bottom case 22 is located on the side facing the target under measurement (test object) when the user wears the apparatus body 100. The top case 21 is disposed on the side (front side) opposite the target under measurement (test object) with respect to the bottom case 22. The detection window 2211 is provided in the rear surface of the bottom case 22, and the biological information measuring module 30 is provided in a position corresponding to the detection window 2211.

The top case 21 may include the barrel 211 and the glass plate 212. In this case, the barrel 211 and the glass plate 212 may be used as an outer wall that protects the internal structure and may allow the user to view information displayed in a display section, such as a liquid crystal display (LCD 70) provided immediately below the glass plate 212, via the glass plate 212. That is, in the biological information measuring apparatus 2 according to the present embodiment, the LCD 70 may be used to display a variety of pieces of information, such as detected biological information, information representing the state of the user's motion, or time information, and present the displayed information to the user via the top case 21. In the above description, a top plate portion of the biological information measuring apparatus 2 is achieved by the glass plate 212 by way of example, and the top plate portion can instead be made of a transparent plastic material or any other non-glass material that forms a transparent member that allows the user to view the LCD 70 and has strength high enough to be capable of protecting the configuration contained in the interior of the top case 21 and the bottom case 22, such as the LCD 70.

The apparatus body 100 includes, in addition to the top case 21 and the bottom case 22, the module substrate 35 as the second substrate, the biological information measuring module 30, which is connected to the module substrate 35, the circuit substrate 40 as the first substrate, the panel frame 42, the circuit case 44, an atmospheric pressure sensor 50 and the geomagnetism sensor 55 as an example of the sensor section, the secondary battery 60, the LCD 70, a vibrator (vibration motor) 80, and a GPS antenna as shown in FIG. 9. It is, however, noted that the configuration of the biological information measuring apparatus 2 is not limited to the configuration shown in FIG. 9; another configuration can be added, and part of the configuration can be omitted. For example, the GPS antenna 90 may be omitted from the configuration shown in FIG. 9.

The biological information measuring module 30 includes a photoelectric sensor. The biological information measuring module 30 including the photoelectric sensor includes at least the first light emitting unit 311, the second light emitting unit 312, and the light receiver 315, as in the first embodiment. The biological information measuring module 30 has the same configuration as that in the first embodiment, and no description of the configuration of the biological information measuring module 30 will therefore be made in the second embodiment.

The circuit substrate 40 has the panel frame 42, which guides the LCD 70 or any other display panel, disposed on one surface of the circuit substrate 40 and further has the circuit case 44, which guides the secondary battery 60 and other components, disposed on the other surface of the circuit substrate 40. The circuit substrate 40 is formed, for example, of a substrate made of an epoxy-based material containing glass fibers, and a wiring pattern formed, for example, of a copper foil is formed on each of the surfaces of the circuit substrate 40. The panel frame 42 and the circuit case 44 are made of polyacetal, polycarbonate, or any other resin.

On the circuit substrate 40 are mounted elements that form a circuit that drives the photoelectric sensor (biological information measuring module 30) to cause it to measure the pulse, a circuit that drives the LCD 70, a circuit that controls the circuits described above, and other circuits. Electrodes to be connected to the LCD 70 are formed on the one surface of the circuit substrate 40 and electrically continuous with electrodes of the LCD 70 via a connector that is not shown. The LCD 70 displays measured pulse data such as the pulse rate, time information such as the current time, and other pieces of information in accordance with each mode of the biological information measuring apparatus 2. Although not shown, as other examples of the sensor section, an acceleration sensor, an angular velocity sensor, a temperature sensor, and other sensors may be disposed on the circuit substrate 40. Further, although not shown, a communication antenna and other electric parts may be disposed on the circuit substrate 40.

The circuit case 44 accommodates the secondary battery 60 (lithium secondary battery), which is a rechargeable battery. The positive and negative terminals of the secondary battery 60 are connected to the circuit substrate 40, and the secondary battery 60 supplies electric power to a circuit that controls the electric power. The electric power is converted into predetermined voltage or otherwise processed by the circuit and supplied to each of the circuits described above to operate the circuit that drives the photoelectric sensor to cause it to measure the pulse, the circuit that drives the LCD 70, the circuit that controls the circuits described above, and other circuits. The secondary battery 60 is charged via a pair of charge terminals electrically continuous with the circuit substrate 40 via an electrically conducting member (not shown) such as a coil spring. In the above description, the secondary battery 60 is used as a battery by way of example, and the battery may instead be a primary battery, which does not need to be charged.

The atmospheric pressure sensor 50 is inserted into a through hole provided in the rear surface of the bottom case 22. The through hole communicates with an outside air area outside the case. That is, a vent for the atmospheric pressure sensor 50 is disposed in a position shifted from the circuit substrate 40 toward the bottom case 22. The outside air area opens toward the user worn portion of the bottom case 22 and toward the portion where the band section (not shown) is connected to the bottom case 22. The atmospheric pressure sensor 50 can acquire, for example, atmospheric pressure data by using the outside air area and the vent as a pressure introduction path (path along which outside air and the like are introduced). The biological information measuring apparatus 2 can therefore provide information on altitude (height above sea level) of the location where the user (wearer) is present (current position) on the basis of the atmospheric pressure data acquired by the atmospheric pressure sensor 50.

The geomagnetism sensor 55 can acquire geomagnetism data on measured orientation of the magnetic field in geomagnetism. The biological information measuring apparatus 2 can therefore present orientation information (position information) at the location where the user is present, for example, to the LCD 70 on the basis of the geomagnetism data acquired by the geomagnetism sensor 55.

As described above, the biological information measuring apparatus 2 according to the present embodiment includes the secondary battery 60, which is accommodated in the case section 20, and the circuit substrate 40, which is electrically connected to the biological information measuring module 30, as shown in FIG. 9. The secondary battery is disposed between the circuit substrate 40 and the biological information measuring module 30. The circuit substrate 40 may be a substrate on which a processor of the biological information measuring apparatus 2 is mounted. The secondary battery 60 and the circuit substrate 40 may be provided in a central portion of the biological information measuring apparatus 2 in a plan view viewed from the side facing the bottom case surface that comes into contact with the test object.

The biological information measuring apparatus 2 may further be provided with the vibrator 80 (vibration motor) between the secondary battery 60 and the top case 21/bottom case 22 in a plan view of the bottom case 22 viewed in the direction perpendicular to the bottom case surface that comes into contact. with the test object. The direction perpendicular to contact surface may be the direction DR1 from the bottom case 22 toward the top case 21 or the direction opposite the direction DR1. In other words, the plan view described above refers to a state in which the bottom case 22 is viewed from the side facing the test object. The vibrator 80 may a component that notifies the user of some kind of notification representing, for example, a result of the measurement performed by the biological information measuring module 30 in the form of vibration and can be used as a user interface different from the LCD 70. In the example shown in FIG. 9, the vibrator 80 is provided in a position shifted from the secondary battery 60 toward the right end in FIG. 9. The vibrator 80 is disposed in a position where it does not overlap with the reflectors 3110 (photoelectric sensor) in a plan view, as shown in FIG. 10.

When the vibrator 80 and the reflectors 3110 (photoelectric sensor) are so disposed as not to overlap with each other as described above, a situation in which vibration produced by the vibrator 80 directly propagates to the reflectors 3110 can be avoided. A situation in which irregular light reflection occurs due to vibration of the reflectors 3110 and hence the test object irradiation efficiency decreases can therefore avoided.

It is preferable that the vibrator 80 is disposed in a position where it does not overlap with the light emitters (first light emitter 3111 and second light emitter 3112) (photoelectric sensor) shown in FIG. 10 in the plan view described above.

When the vibrator 80 is disposed in a position where it does not overlap with the light emitters (first light emitter 3111 and second light emitter 3112) (photoelectric sensor) in the plan view as described above, a situation in which the vibration produced by the vibrator 80 propagates to the reflectors 3110 can be avoided. The suppression of the vibration propagation can suppress a decrease in the test object irradiation efficiency due to variation in light emission state resulting from vibration of the light emitters (first light emitter 3111 and second light emitter 3112).

Further, in the biological information measuring apparatus 2, the light emitters (first light emitter 3111 and second light emitter 3112) are preferably disposed between the geomagnetism sensor 55 and the vibrator 80 in the plan view of the bottom case 22 viewed in the direction perpendicular to the bottom case surface that comes into contact with the test object.

When the light emitters (first light emitter 3111 and second light emitter 3112) are disposed between the geomagnetism sensor 55 and the vibrator 80 as described above, the distance between the geomagnetism sensor 55 and the vibrator 80 can be increased. Since the geomagnetism sensor 55 tends to be affected by the magnetism emitted from the vibrator 80, increasing the distance between the geomagnetism sensor 55 and the vibrator 80 allows reduction in influence of the magnetism emitted from the vibrator 80 on the geomagnetism sensor 55. Therefore, even in the compact biological information measuring apparatus 2 having a restricted size, such as a wrist apparatus as large as a wristwatch, the influence of the magnetic noise on the geomagnetism sensor 55 can by suppressed, whereby the geomagnetism can be stably detected.

Examples of preferable arrangement of the constituent parts in the apparatus body 100 of the biological information measuring apparatus 2 other than the arrangement example described above will be described below with reference also to the plan arrangement diagram shown in FIG. 10. FIG. 10 schematically shows the circuit substrate 40 in a plan view viewed in the first direction DR1 shown in FIG. 9.

An example of the arrangement of the atmospheric pressure sensor 50 will first be described. The atmospheric pressure sensor 50 is preferably disposed in a position where it does not overlap with the light emitters (first light emitter 3111 and second light emitter 3112) in the plan view viewed from the side facing the test object, as shown in FIG. 10.

The light emitters (first light emitter 3111 and second light emitter 3112) need to be so disposed as to face the test object. Further, the atmospheric pressure sensor 50, which detects the atmospheric pressure, needs to be provided with a vent that communicates with the atmosphere outside the case.

The light emitters (first light emitter 3111 and second light emitter 3112) and the atmospheric pressure sensor 50 need to be so located as to face the test object outside the case, as described above. When the atmospheric pressure sensor 50 and the light emitters (first light emitter 3111 and second light emitter 3112) are so located in positions where they do not overlap with each other in the plan view, they are allowed, in their positions, to face the test object (target) and a vent can be provided, whereby the thickness of the apparatus can be reduced.

Further, the configuration in which the vent for the atmospheric pressure sensor 50 and the light emitters (first light emitter 3111 and second light emitter 3112) are separate from each other by a large distance in the plan view can suppress the influence on the measurement resulting from a situation in which external light (ambient light) enters the apparatus through the vent is mixed with the light emitted from the light emitters (first light emitter 3111 and second light emitter 3112).

An example of the arrangement of an acceleration sensor 66 will next be described. The acceleration sensor 66 is preferably disposed in a position opposite the vibrator 80 with respect to the light emitters (first light emitter 3111 and second light emitter 3112) in the plan view viewed from the side facing the test object, as shown in FIG. 10.

When the light emitters (first light emitter 3111 and second light emitter 3112) are disposed between the acceleration sensor 66 and the vibrator 80 as described above, the distance between the acceleration sensor 66 and the vibrator 80 can be increased. Since the acceleration sensor 66 tends to be affected by the vibration produced by the vibrator 80, increasing the distance between the acceleration sensor 66 and the vibrator 80 allows reduction in influence of the vibration produced by the vibrator 80 on the acceleration sensor 66. Therefore, even in the compact biological information measuring apparatus 2 having a restricted size, such as a wrist apparatus as large as a wristwatch, the acceleration can be stably detected.

An antenna for communication (communication antenna) 43, the GPS antenna 90, and other antennas are also preferably disposed in positions where they do not overlap with the light emitters (first light emitter 3111 and second light emitter 3112) in the plan view viewed from the side facing the test object, as shown in FIG. 10. The relatively large constituent parts can therefore be disposed in positions where they do not overlap with each other, whereby the thickness of the apparatus can be reduced.

Further, when the geomagnetism sensor 55, the antenna for communication (communication antenna) 43, and the GPS antenna 90, which tend to be affected, for example, by a metal plate, are disposed in the same area as shown in FIG. 10, a reinforcing plate made of a metal that reinforces the case (top case 21 and bottom case 22) or any other enclosure that accommodates the components described above and a decorative plate (bezel, for example) that improves the exterior appearance of the case can be arranged with increased layout flexibility, whereby the resultant efficient arrangement allows further size reduction.

### 7. Configuration Biological Information Measuring Apparatus According to Third Embodiment

A biological information measuring apparatus according to a third embodiment of the invention will next be described with reference to the drawings. The biological information measuring apparatus according to the third embodiment is worn by a biological body (human body, for example) from which biological information is measured, as in the first embodiment described above, and is a heart rate monitoring apparatus that measures biological information, such as the pulse rate (heart rate). In the following drawings, to allow each component to be large enough to be recognizable in the drawings, the dimension and scale of the component differ from values of an actual component as appropriate in some cases.

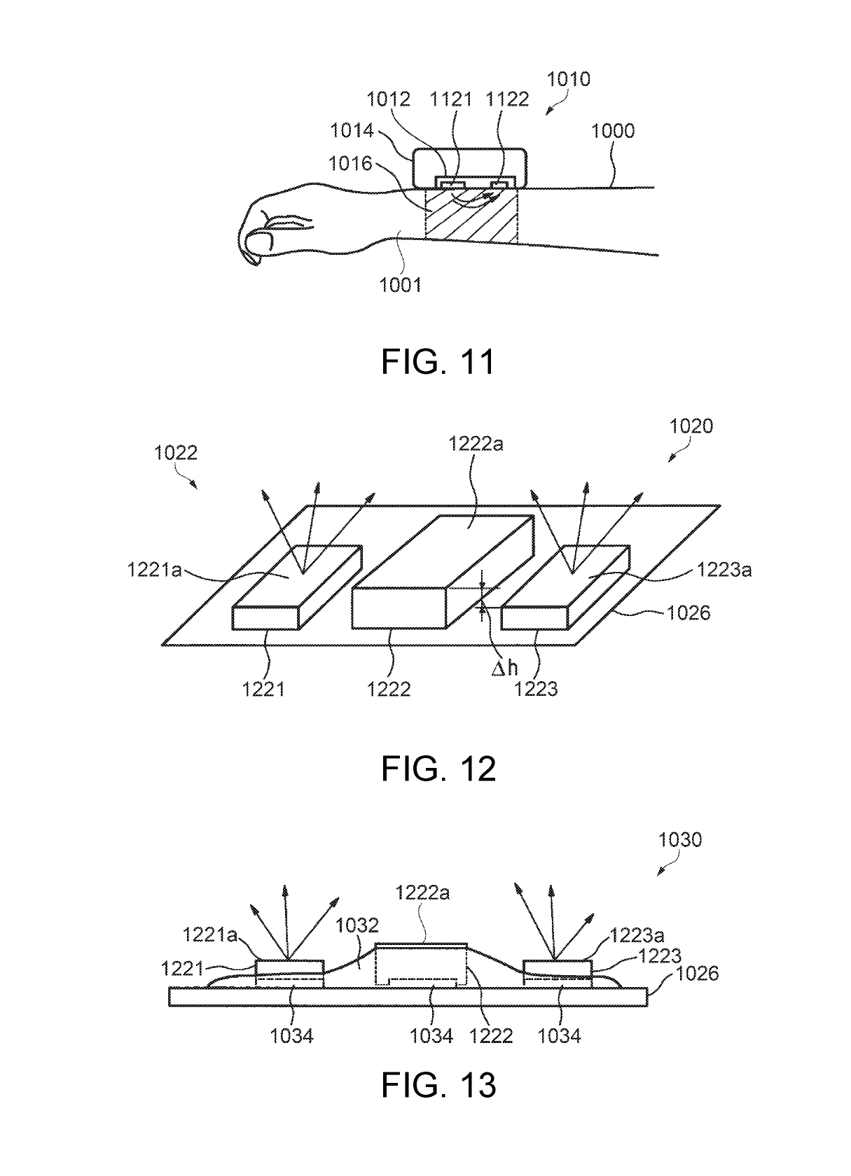

Before a heart rate monitoring apparatus 1010 as the biological information measuring apparatus according to the third embodiment is described, a heart rate monitoring apparatus of related art as the heart rate monitoring apparatus according to the third embodiment will be described with reference to FIG. 11.

FIG. 11 is a cross-sectional view of the heart rate monitoring apparatus 1010 as a biological information measuring apparatus of related art that measures a physiological parameter (biological information) of a user (subject) 1000 who wears the heart rate monitoring apparatus (FIG. 11 shows user's arm). The heart rate monitoring apparatus 1010 includes a sensor 1012, which measures the heart rate as at least one physiological parameter of the user 1000, and a case 1014, which accommodates the sensor 1012. The heart rate monitoring apparatus 1010 is worn around an arm 1001 of the user 1000 with the aid of a fixing section 1016 (band, for example).

The sensor 1012 is a heart rate monitoring sensor including light emitting devices 1121, which serve as light emitters formed of two sensor elements, and a light receiving device 1122, which serves as a light receiver, for measuring or monitoring the heart rate. The sensor 1012 may instead be a sensor that measures one or more physiological parameters (heart rate, blood pressure, respiratory volume, skin conductivity, and skin humidity, for example). In a case where the case 1014 includes a band-type housing, the sensor 1012 can be used as a wristwatch-shaped monitoring apparatus used, for example, in sports. The case 1014 may have any shape that can hold the sensor 1012 in a desired position mainly on the user 1000 and may further optionally accommodate a battery, a processing unit, a display, a user interface, and other elements.

The biological information measuring apparatus of related art is the heart rate monitoring apparatus 1010 for monitoring the user's heart rate. The sensor 1012 is an optical sensor formed of the light emitting devices 1121 and the light receiving device 1122. The optical heart rate monitoring using the optical sensor depends on the light emitting devices 1121 (LEDs are typically used) as a light source that applies light onto the skin. The light radiated from the light emitting devices 1121 to the skin is partly absorbed by the blood flowing through blood vessels under the skin, and the remainder of the light is reflected off the blood and exits out of the skin. The reflected light is then captured by the light receiving device 1122 (photodiode is typically used). A light reception signal from the light receiving device 1122 is a signal containing information corresponding to the amount of blood flowing through the blood vessels. The amount of blood flowing through the blood vessels changes when the heart pulses. The signal from the light receiving device 1122 then changes in correspondence with the heartbeats. That is, the change in the signal from the light receiving device 1122 corresponds to the heart rate pulses. Counting the number of pulses per unit time (per 10 seconds, for example) allows the number of heartbeats in one minute (that is, heart rate) to be obtained.

A heart rate monitoring apparatus 1020 as the biological information measuring apparatus according to the third embodiment will be described below with reference to FIG. 12. FIG. 12 is a perspective view showing the heart rate monitoring apparatus as the biological information measuring apparatus according to the third embodiment. Although not shown in FIG. 12, the heart rate monitoring apparatus 1020 as the biological information measuring apparatus according to the third embodiment is worn around the user's arm with the aid of a fixing section, such as a band section, as in the first embodiment described above.

The heart rate monitoring apparatus 1020 as the biological information measuring apparatus according to the third embodiment includes light emitting devices 1221 and 1223 as a plurality of (two in present example) light emitters and a light receiving device 1222 as a single light receiver, which are arranged in a single row. Specifically, the heart rate monitoring apparatus 1020 includes a sensor 1022 including at least two sensor elements (in the present example, the two light emitting devices 1221 and 1223, which serve as a first light emitter and a second light emitter, and the light receiving device 1222, which serves as a light receiver, are used as three sensor elements). Although not shown, the same light blocking walls 318 and 319 (see FIG. 7) as those in the variation of the first embodiment described above are desirably provided between the light receiving device 1222 and the light emitting device 1221 and between the light receiving device 1222 and the light emitting device 1223, respectively.

The light receiving device 1222 as the light receiver is disposed between the two light emitting devices 1221 and 1223 as the first and second light emitters. The two light emitting devices 1221 and 1223 as the first and second light emitters are disposed in positions symmetric with respect to an imaginary line passing through the center of the light receiving device 1222 as the light receiver. Arranging the light emitting devices 1121, 1123 and the light receiving device 1222 as described above allows a dead space to be reduced and space saving to be achieved. Further, the light fluxes emitted from the first and second light emitters located in axially symmetric positions are summed on the light receiver for more accurate detection.

The sensor elements allow detection of a sensor signal. The sensor 1022 includes the optical sensor formed of the light emitting devices 1121 and 1223 using two LEDs for emitting light toward the user s skin and the at least one light receiving device 1222 (photodiode) for receiving light reflected off the skin. The heart rate monitoring apparatus 1020 further includes a case or a housing (not shown). The case or the housing may be similar to or the same as the case 1014 shown in FIG. 11 or may be similar to or the same as the case section 20 in the first embodiment described above.

The sensor 1022 is carried by one surface of a carrier (substrate) 1026. The configuration including the carrier (substrate) 1026 and the sensor 1022 carried on the carrier (substrate) 1026 corresponds to the biological information measuring module. The same holds true for the following fourth to sixth embodiments. The light emitted from the light emitting devices 1221 and 1223 is not absorbed by the skin or other body sites but is reflected off the skin and other body sites and can directly reach the light receiving device 1222. In the heart rate monitoring apparatus 1020, the distance between the carrier 1026 and the upper surfaces 1221a and 1223a of the light emitting devices 1221 and 1223 is smaller than the distance between the carrier 1026 and the upper surface 1222a of the light receiving device 1222. That is, the distance between the carrier 1026 and the upper surfaces 1221a and 1223a of the. light emitting devices 1221 and 1223 differs from the distance between the carrier 1026 and the upper surface 1222a of the light receiving device 1222 by Δh. The light receiving device 1222 receives light through the upper surface 1222a thereof, which is the uppermost surface layer. The configuration described above advantageously allows most of the light emitted from the light emitting devices 1221 and 1223 to travel toward the skin and the reflected light to be directly incident on the light receiving device 1222, for example, via no air layer. In other words, since the structure in which the light receiving device 1222 is in intimate contact with the skin is employed, a structure in which no gap is likely to be created between the upper surface (light reception surface) 1222a of the light receiving device 1222 and the skin can be achieved, whereby a situation in which light that serves as a noise source, such as ambient light, is incident on the upper surface 1222a can be avoided. Further, the light that is emitted from the light emitting devices 1221 and 1223 but does not pass through the skin, for example, the light that is emitted from the light emitting devices 1221 and 1223 and directly incident on the light receiving device 1222 cannot reach the upper surface 1222a of the light receiving device 1222.

### 8. Configuration of Biological Information Measuring Apparatus According to Fourth Embodiment

A heart rate monitoring apparatus 1030 as a biological information measuring apparatus according to a fourth embodiment will next be described with reference to FIG. 13. FIG. 13 is a front view showing the heart rate monitoring apparatus as the biological information measuring apparatus according to the fourth embodiment. Although not shown in FIG. 13, the heart rate monitoring apparatus 1030 as the biological information measuring apparatus according to the fourth embodiment is worn around the user's arm with the aid of a fixing section, such as a band section, as in the first embodiment described above.

Electric connection terminal 1034 of the light emitting devices 1221 and 1223 as the light emitters and the light receiving device 1222 as the light receiver preferably need to be covered with an insulating material (epoxy resin, for example) 1032 for electric element protection, as shown in FIG. 13. The insulting material 1032 can be configured not to cover the light emitting device 1221, 1223 or the light receiving device 1222. Specifically, the insulating material 1032 can be configured to fill the area between the light emitting device 1221 and the light receiving device 1222 and the area between the light emitting device 1223 and the light receiving device 1222. In other words, the insulating material 1032 can be configured not to cover at least the upper surface 1222a of the light receiving device 1222, the upper surface 1221a of the light emitting device 1221, or the upper surface 1223a of the light emitting device 1223. The configuration described above can eliminate interference with the measurement due to an air gap between the skin and the light emitting devices 1221, 1223. The insulating material 1032 may instead be configured to cover the upper surfaces 1221a and 1223a of the light emitting devices 1221 and 1223 and the upper surface 1222a of the light receiving device 1222. The configuration described above allows protection of the upper surface 1222a of the light receiving device 1222, which comes into contact with the skin, and the upper surfaces 1221a and 1223a of the light emitting devices 1221 and 1223, whereby damage of the upper surface 1222a of the light receiving device 1222 and the upper surfaces 1221a and 1223a of the light emitting devices 1221 and 1223 can be avoided. In this case, the insulating material 1032 can also be taken as a protective film.

The heart rate monitoring apparatus 1030 as the biological information measuring apparatus according to the fourth embodiment is provided with the insulating material 1032 made oaf an epoxy resin as a typically feasible example. In FIG. 13, the insulating material 1032 is so disposed as not to cover the upper surface 1221a of the light emitting device 1221 or the upper surface 1223a of the light emitting device 1223 and protects the electric connection terminals 1034. The light emitted from the light emitting devices 1221 and 1223 is indicated by the arrows.

As described above, the area where the insulating material 1032 is arranged is minimized to the extent that the heart rate monitoring apparatus 1030 functions correctly and the electric connection terminals 1034 of the light emitting devices 1221, 1223 and the light receiving device 1222 are protected, whereby the heart rate monitoring apparatus 1030 can be further improved. Although not shown, the same light blocking walls 318 and 319 (see FIG. 7) as those in the variation of the first embodiment described above are further preferably provided between the light receiving device 1222 and the light emitting device 1221 and between the light receiving device 1222 and the light emitting device 1223, respectively.

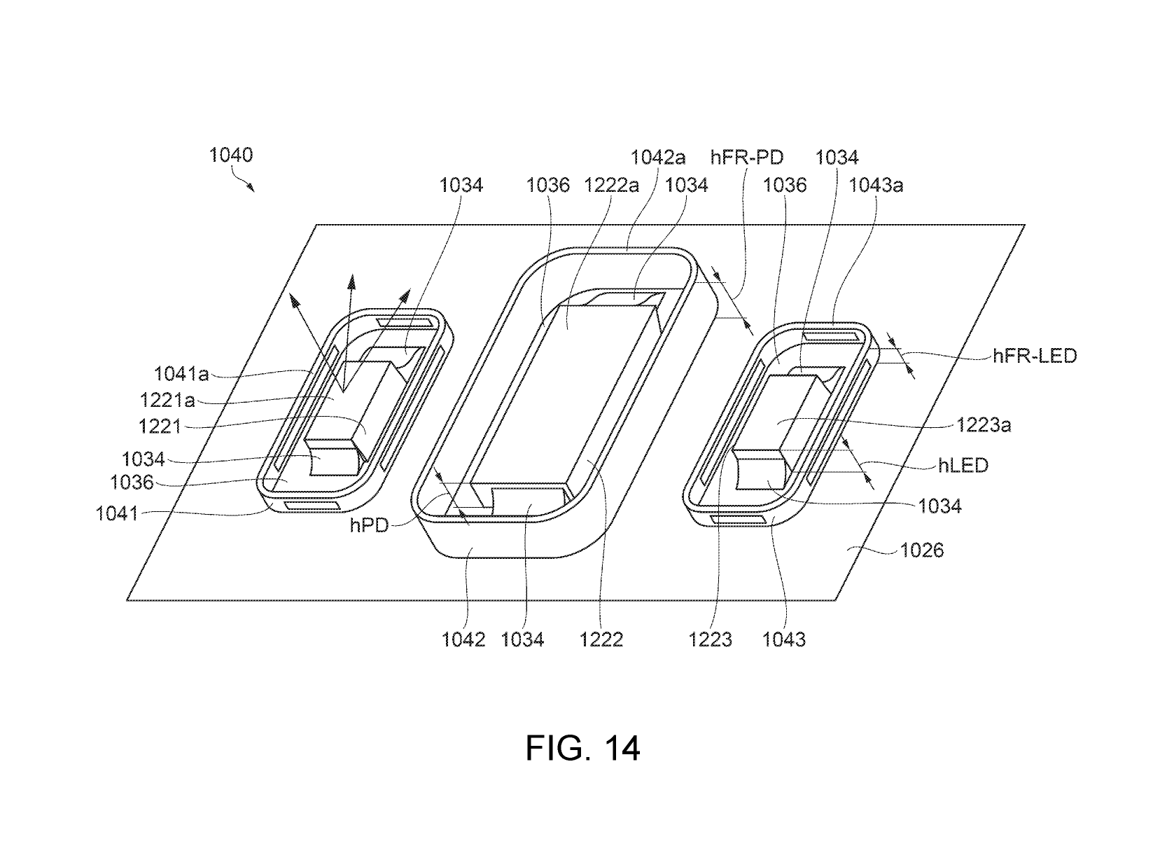

Instead of the configuration in the fourth embodiment in which an epoxy resin is injected, a heart rate monitoring apparatus 1040 as a biological information measuring apparatus according to a fifth embodiment shown in FIG. 14 is further preferably achieved.

### 9. Configuration of Biological Information Measuring Apparatus According to Fifth Embodiment

A heart rate monitoring apparatus 1040 as a biological information measuring apparatus according to a fifth embodiment will next be described with reference to FIG. 14. FIG. 14 is a perspective view showing the heart rate monitoring apparatus as the biological information measuring apparatus according to the fifth embodiment. Although not shown in FIG. 14, the heart rate monitoring apparatus 1040 as the biological information measuring apparatus according to the fifth embodiment is worn around the user's arm with the aid of a fixing section, such as a band section, as in the first embodiment described above.

In the heart rate monitoring apparatus 1040 as the biological information measuring apparatus according to the fifth embodiment, created frames 1041, 1042, and 1043 are disposed. The frames 1041, 1042, and 1043 are disposed around the light emitting devices 1221 and 1223 as the light emitters and the light receiving device 1222 as the light receiver, respectively, and gaps 1036 are formed between the frames 1041, 1042, 1043 and the light emitting devices 1221, 1223/light receiving device 1222. An insulating material (not shown in FIG. 14) is injected by using the frames 1041, 1042, and 1043 as a guide to cover the electric connection terminals 1034 of the light emitting devices 1221, 1223 and the light receiving device 1222.

In the example shown in the fifth embodiment, the light emitting devices 1221, 1223 and the light receiving device 1222 are surrounded by the individual frames 1041, 1042, and 1043. As another example, the frames 1041, 1042, and 1043 may be linked to each other, or all the sensor elements may be surrounded by a unitary frame. The frames 1041, 1042, and 1043 can be used as light blocking walls as an example of wall sections (light blocking sections). Using the frames 1041, 1042, and 1043 as the light blocking walls can prevent the light emitted from the light emitting devices 1221 and 1223 from being directly incident on the light receiving device 1222.

As an improved feature for preventing influence on the function of the heart rate monitoring apparatus 1040, the upper edges 1041a and 1043a of the frames 1041 and 1043 around the light emitting devices 1221 and 1223 are preferably lower than. the upper surfaces 1221a and 1223a of the light emitting devices 1221 and 1223. In other words, the distance hFR-LFD between the upper edges 1041a, 1043a of the individual frames 1041, 1043 and the carrier 1026 is equal to or smaller than the distance hLED between the upper surfaces 1221a, 1223a of the light emitting devices 1221, 1223 surrounded by the individual frames 1041, 1043 and the carrier 1026 (hFR-LED≦hLED).

The difference between the distance hLED between the upper surfaces 1221a, 1223a of the light emitting devices 1221, 1223 and the carrier 1026 and the distance hFR-LED between the upper edges 1041a, 1043a of the frames 1041, 1043 and the carrier 1026 is preferably so set as to fall within a range from 0.1 to 0.8 mm. More preferably, the difference between the distance hLED between the upper surfaces 1221a, 1223a of the light emitting devices 1221, 1223 and the carrier 1026 and the distance hFR--LED between the upper edges 1041a, 1043a of the frames 1041, 1043 and the carrier 1026 is so set as to fail within a range from 0.2 to 0.5 mm.

Further, the upper edge 1042a of the frame (receiver frame) 1042 around the light receiving device 1222 is preferably higher than the upper surface 1222a of the light receiving device 1222. In other words, the distance hFR-LED between the upper edge 1042a of the frame 1042 and the carrier 1026 is greater than the distance hPD between the upper surface 1222a of the light receiving device 1222 surrounded by the frame 1042 and the carrier 1026 (hFR-LED>hPD).

The difference between the distance hPD between the upper surface 1222a of the light receiving device 1222 and the carrier 1026 and the distance hFR-LED between the upper edge 1042a of the frame 1042 and the carrier 1026 is preferably so set as to fall within a range from 0 to 0.5 mm. More preferably, the difference between the distance hPD between the upper surface 1222a of the light receiving device 1222 and the carrier 1026 and the distance hFR-LED between the upper edge 1042a of the frame 1042 and the carrier 1026 is so set as to fall within a range from 0.1 mm to 0.2 mm.

Further, the distance hFR-PD between the upper edge 1042a of the frame 1042 and the carrier 1026 is greater than the distance hLED between the upper surfaces 1221a, 1223a of the light emitting devices 1221, 1223 and the carrier 1026 (hFR-PD>hLED).

For example, in a case where the light receiving device 1222 is disposed closely to the light emitting devices 1221 and 1223, only a single frame wall may be present between the light receiving device 1222 and the light emitting device 1221 and between the light receiving device 1222 and the light emitting device 1223. The configuration described above is employed in some cases because it simplifies the manufacture of the apparatus. In a case where the two single frame walls form a case, the frame wall of the frame around the light receiving device 1222 coincides with the frame walls of the frames around the light emitting devices 1221 and 1223. This means that the height of the frame walls around the light emitting devices 1221 and 1223 increases. In detail, the height of one frame wall of each of the frames 1041 and 1043, which surround the light emitting devices 1221 and 1223, the height of the frame wall on the side where the light receiving device 1222 is present increases, and the other frame wall is lower than the corresponding one of the upper surfaces of the 1221a and 1223a of the light emitting devices 1221 and 1223.

Further, in place of the frames 1041, 1042, and 1043, first wall sections may be provided between the light receiving device 1222 and the light emitting devices 1221, 1223, and second wall sections may be provided in positions outside the light emitting devices 1221 and 1223, that is, on the side opposite the first wall sections with respect to the light receiving device 1222.

When the configuration described above is employed, the distance between the carrier 1026 and the upper surfaces of the first wall sections may be so set as to be greater than the distance between the carrier 1026 and the upper surfaces of the second wall sections. When the setting described above is employed, the function of the frames can be achieved by a smaller number of members than in the case where the light emitting devices and the light receiving device are surrounded by the frames as shown in FIG. 14.

When the frames 1041, 1043, and 1042 are used as in the fifth embodiment, a situation in which the injected insulating material, such as an epoxy resin, flows out can be avoided. Further, creating the additional structure to separate the insulating material, such as an epoxy resin, from the other portions as described above is an option that allows high volume productivity. The frames 1041, 1043, and 1042 may be made of the same material as that of the carrier 1026. For example, the frames may be formed in injection molding using an epoxy-based resin or a polycarbonate-based resin. Further, the frame 1042 may be connected to at least one of the frames 1041 and 1043. This configuration allows reduction in material cost.

As described above, the insulating material 1032 (see FIG. 13) protects the electric connection terminals 1034 of the sensor elements (light emitting devices 1221, 1223 and light receiving device 1222). The electric connection terminals 1034, however, need to be further in contact with an additional electronic apparatus (driver, detection electronics, processor, or power supply) that is another element. This further means that the carrier 1026 (or may be printed circuit board (PCB)) is somehow electrically connected to the additional electronic apparatus. Further, the structure of the heart rate monitoring apparatus according to the present embodiment is also applicable not only to a measurement apparatus that measures the heart rate but also to a measurement apparatus that measures the pulse waves or pulse rate.

### 10. Configuration of Biological Information Measuring Apparatus According to Sixth Embodiment

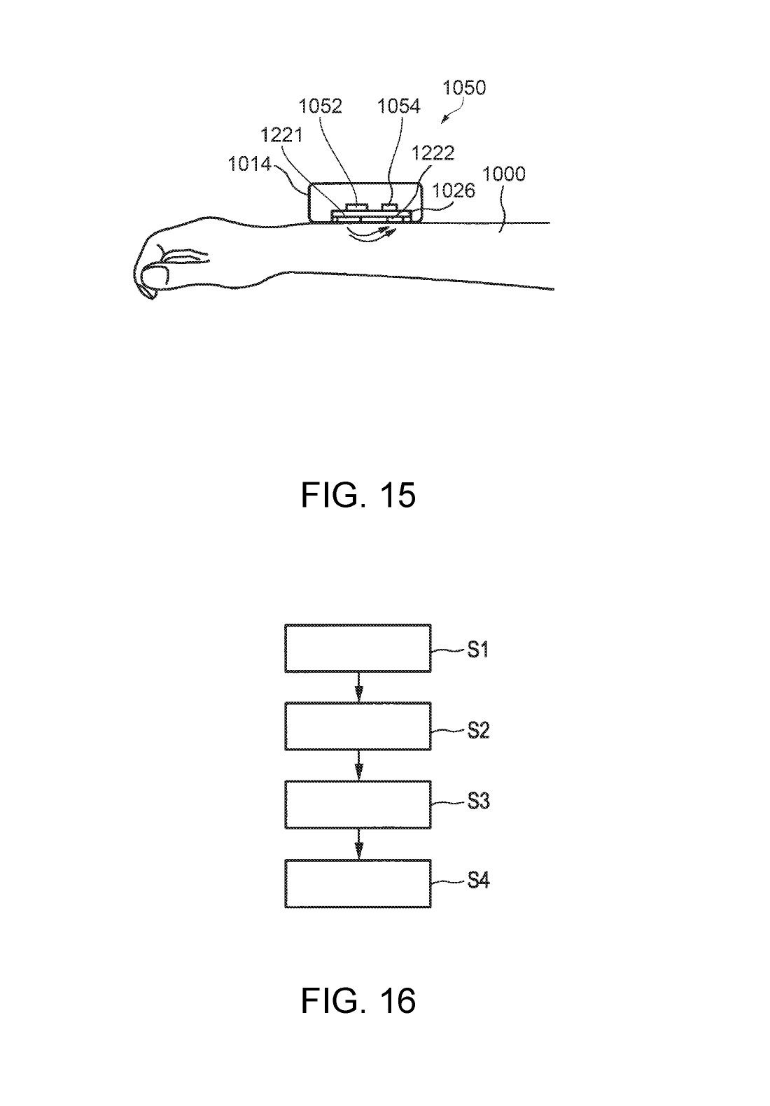

A heart rate monitoring apparatus 1050 as a biological information measuring apparatus according to a sixth embodiment will be described with reference to FIG. 15. FIG. 15 is a cross-sectional view showing the heart rate monitoring apparatus as the biological information measuring apparatus according to the sixth embodiment. Although not shown in FIG. 15, the heart rate monitoring apparatus 1050 as the biological information measuring apparatus according to the sixth embodiment is worn around the user's arm with the aid of a fixing section, such as a band section, as in the first embodiment described above.

The heart rate monitoring apparatus 1050 as the biological information measuring apparatus according to the sixth embodiment includes the additional electronic apparatus described above (processor 1052 and driver 1054, for example). External electric connection terminals of the additional electronic apparatus (not shown) are not disposed on the carrier 1026 on which the sensor elements (light emitting device 1221 as light emitter and light receiving device 1222 as light receiver) are disposed. That is, the additional electronic apparatus are disposed on a carrier or a substrate different from the carrier or substrate on which the sensor elements are disposed. The configuration described above allows a necessary additional electronic apparatus to be incorporated in the heart rate monitoring apparatus 1050 with satisfactory contact between the skin and the sensor elements (light emitting device 1221 and light receiving device 1222) maintained. For example, the external electric connection terminals can be disposed on the side surface of the carrier 1026.

As described above, different types of sensors can be used in the biological information measuring apparatus according to the present embodiment of the invention. For example, in a case where the light receiving device 1222 described above is an electric sensor, two skin conductance electrodes (for example, sensor elements (light emitting device 1221 and light receiving device 1222 shown in FIG. 12)) that come into contact with the user's skin and measure the user's conductivity are covered by the skin. Further, two or more types of additional sensor can be used in a biological information measuring apparatus of this type. Moreover, an arbitrary number of sensor elements can be used.

FIG. 16 is a flowchart of a method for manufacturing the biological information measuring apparatus that measures a proposed physiological parameter in the third to sixth embodiments.

In first step S1, the sensor 1022 formed of at least two sensor elements (light emitting device 1221 and light receiving device 1222) for detecting a sensor signal is disposed on the carrier 1026. In second step S2, electrical contacts of the sensor elements described above are formed on the carrier 1026. In third step S3, one or more of the frames 1041 and 1042 are formed on the carrier 1026 and around the sensor 1022 and/or the individual sensor elements (light emitting device 1221 and light receiving device 1222). In fourth step S4, the insulating material 1032 is injected into the area surrounded by the frames 1041 and 1042 that cover none of the upper surfaces 1221a and 1223a of the sensor elements (light emitting device 1221 and light receiving device 1222) provided on the carrier 1026 so that the area is filled with the insulating material 1032.

The third to sixth embodiments described above propose a method for achieving electrical contact protection that does not adversely affect the performance of the biological information measuring apparatus. The protection is formed in a method that maintains the performance of the sensor. For example, at least one of the frames 1041 and 1043 prevents shift of the position of the sensor with respect to the skin. Further, at least one of the frames 1041 and 1043 can serve to prevent the emitted light from directly impinging on the light receiving device 1222. The height of one side of the frames 1041 and 1043 around the light emitting devices 1221 and 1223, the side facing the light receiving device 1222, needs to be smaller than the height of the upper surfaces 1221a and 1223a of the light emitting devices 1221 and 1223. In addition, the frame 1042 around the light receiving device 1222 may be higher than the upper surface 1222a of the light receiving device 1222.

The configuration of the gap between each of the light emitting devices and the light receiving device described in the first embodiment can be applied also to the biological information measuring apparatus according to the third to sixth embodiments described above. The same advantageous effects provided by the first embodiment can be provided by the configuration described above.

The embodiments of the invention have been described above in detail, and a person skilled in the art will readily appreciate that a large number of variations are conceivable to the extent that they do not substantially depart from the novel items and advantageous effects of the invention. Such variations are all therefore assumed to fall within the scope of the invention. For example, a term described at least once in the specification or the drawings along with a different term having a boarder meaning or the same meaning can be replaced with the different term anywhere in the specification or the drawings. Further, the configuration and operation of the biological information measuring apparatus are not limited to those described in the embodiments of the invention, and a variety of changes can be made thereto.

What is claimed is:

1. A biological information measuring module comprising:
a light emitter;
a reflector that has a tapered section and reflects light; and
a light receiver that receives reflected light that is the light reflected off a target.

2. The biological information measuring module according to claim 1, wherein a height of the reflector along a direction in which the light exits is smaller than or equal to 770 μm but greater than or equal to 200 μm.

3. The biological information measuring module according to claim 1, wherein a height of the reflector is smaller than or equal to 700 μm but greater than or equal to 200 μm.

4. The biological information measuring module according to claim 1, wherein a height of the reflector is smaller than or equal to 650 μm but greater than or equal to 200 μm.

5. The biological information measuring module according to claim 1, wherein the reflector has a circular shape in a plan view viewed from the target.

6. The biological information measuring module according to claim 1, wherein the reflector has a polygonal shape in a plan view.

7. The biological information measuring module according to claim 1, wherein the reflector is higher than the light receiver.

8. The biological information measuring module according to claim 1, wherein the reflector is lower than the light receiver.

9. The biological information measuring module according to claim 1, wherein the light emitter is formed of a plurality of light emitters.

10. The biological information measuring module according to claim 9, wherein the plurality of light emitters are arranged in positions symmetric with respect to an imaginary line passing through a center of the light receiver.

11. The biological information measuring module according to claim 1, wherein the reflector is provided with a reflection film.

12. The biological information measuring module according to claim 1, wherein the reflector includes a guide section disposed in a position shifted from the tapered section toward the target.

13. The biological information measuring module according to claim 1, wherein in a plan view, a distance between the reflector and the light receiver is smaller than a width of the reflector in a direction in which the reflector and the light receiver are arranged.

14. The biological information measuring module according to claim 1, wherein a light blocking wall is disposed between the light emitter and the light receiver.

15. A biological information measuring apparatus comprising
the biological information measuring module according to claim 1;
a first substrate to which a sensor section is connected; and
a second substrate to which at least the light emitter, the reflector, and the light receiver contained in the biological information measuring module are connected,

16. A biological information measuring apparatus comprising:
the biological information measuring module according to claim 2;
a first substrate. to which a sensor section is connected; and
a second substrate to which at least the light emitter, the reflector, and the light receiver contained in the biological information measuring module are connected.

17. The biological information measuring apparatus according to claim 15, further comprising
a vibrator that notifies a result of measurement performed by the biological information measuring module in a form of vibration,
wherein the vibrator is disposed in a position where the vibrator does not overlap with the reflector in a plan view viewed from the target.

18. The biological information measuring apparatus according to claim 15, further comprising
a vibrator that notifies a result of measurement performed by the biological information measuring module in a form of vibration,
wherein the vibrator is disposed in a position where the vibrator does not overlap with the light emitter in a plan view viewed from the target.

19. The biological information measuring apparatus according to claim 15,
wherein the sensor section includes an atmospheric pressure sensor, and
the atmospheric pressure sensor is disposed in a position where the atmospheric pressure sensor does not overlap with the light emitter in a plan view viewed from the target.

20. The biological information measuring apparatus according to claim 16,
wherein the sensor section further includes a geomagnetism sensor, and
the light emitter is disposed in a position between the geomagnetism sensor and the vibrator in the plan view viewed from the target.
